(19) United States
(12) Patent Application Publication
(10) Pub. No.: US 2015/0011892 A1
(43) Pub. Date: Jan. 8, 2015

(54) METHODS FOR EVALUATING TISSUE INJURIES

(71) Applicant: Harvard Apparatus Regenerative Technology, Inc., Holliston, MA (US)

(72) Inventor: Ron Sostek, Newton, MA (US)

(21) Appl. No.: 14/373,215

(22) PCT Filed: Jan. 20, 2013
(86) PCT No.: PCT/US2013/022346

§ 371 Date: Jul. 18, 2014

Related U.S. Application Data
(60) Provisional application No. 61/589,292, filed on Jan. 20, 2012.
(60) Provisional application No. 61/637,266, filed on Apr. 23, 2012.
(60) Provisional application No. 61/638,481, filed on Apr. 25, 2012.

(51) Int. Cl. A61B 5/00

(52) U.S. Cl. CPC A61B 5/445; A61B 5/0077; A61B 5/0064
USPC 600/473

(57) ABSTRACT

Aspects of the invention relate to methods and devices for evaluating tissue injuries. In some embodiments, the invention provides methods and devices for evaluating tissue injuries based on infrared emission spectra and/or emission levels from injured tissue. In some embodiments, the invention provides methods and devices for evaluating skin injuries (e.g., skin burns) and for targeting treatment delivery to injuries including skin injuries.

### RELATED APPLICATIONS

This application claims priority under 35 U.S.C. §119(e) from U.S. Provisional Application Ser. No. 61/589,292 having a filing date of Jan. 20, 2012 and entitled “METHODS FOR EVALUATING TISSUE INJURIES,” from U.S. Provisional Application Ser. No. 61/637,266 having a filing date of Apr. 23, 2012 and entitled “METHODS FOR EVALUATING TISSUE INJURIES,” and from U.S. Provisional Application Ser. No. 61/638,481 having a filing date of Apr. 25, 2012 and entitled “METHODS FOR EVALUATING TISSUE INJURIES,” the contents of each of which are incorporated herein in their entirety by reference.

### BACKGROUND

A burn is a type of injury to skin or another tissue that can be caused by a variety of different insults, including, for example, heat, electricity, chemicals, light, radiation or friction. It is often important to determine the extent of a burn injury because, depending on its severity, a burn may be treated with first aid without the need for hospitalization or professional medical care or may require more specialized treatment such as those available at specialized burn centers. Furthermore, managing burn injuries properly is important because they are common, painful and can result in disfiguring and disabling scarring, amputation of affected parts or death in severe cases. The treatment of burns may include the removal of dead tissue (debridement), applying dressings to the wound, fluid resuscitation, administering antibiotics, and skin grafting, depending on the depth and severity of the burn.

Similarly, appropriate treatments for other injuries or wounds (including trauma injuries, contusions, cuts, abrasions, etc.) depend on the extent and severity of the injured or wounded tissue. However, it is often difficult to assess the extent or severity of a tissue injury immediately after the injury occurs. It can take several days for the full extent of an injury to become apparent through visible physical changes of the injured tissue. This delay can make it difficult to select the most appropriate treatment soon after the injury occurs.

### SUMMARY OF THE INVENTION

In some embodiments, aspects of the invention relate to methods and devices for evaluating tissue injuries by analyzing emission spectra and/or emission levels from injured tissue. In some embodiments, infrared emissions from a tissue region can be used to evaluate the presence and/or extent of an injury in the tissue region. According to aspects of the invention, the level of infrared (IR) emission from a tissue is affected by physical changes in the tissue that can be caused by injury. In some embodiments, an injury is a burn. In some embodiments, an injury is a traumatic injury. In some embodiments, an injury is a cut, a tear, an abrasion, or other physical disruption of tissue integrity, for example due to chemical exposure, radiation, friction, and/or natural disease changes. In some embodiments, an injury can result from a pressure wave (e.g., from an explosion or other source of pressure blast).

In some embodiments, emissions (e.g., IR emissions) from an injured tissue can be used to determine the extent or severity of an injury soon after the injury occurs (e.g., within several minutes to several hours of the injury), and long before the full extent or severity of the injury can be determined from visible tissue changes. In some embodiments, relative emission intensities (e.g., relative IR emission intensities) from different areas of an injured tissue can be used to identify different levels of injury severity within the injured tissue. In some embodiments, the rate of change of emission intensity as a function of distance within an injured tissue can be used to determine different levels of injury within the tissue. It should be appreciated that emission intensities (e.g., relative emission intensities) can be analyzed using any suitable method. In some embodiments, emission levels are analyzed and/or displayed (e.g., using a color display), in some embodiments, changes in emission levels are analyzed and/or displayed. Methods described herein can be useful to identify areas of severe injury (e.g., deep burns, third degree burns, deep or severe blunt force injury, etc.), areas of minor injury (e.g., shallow burns, first degree burns, mild bruising or contusion, etc.), or areas of intermediate injury (e.g., second degree burn). In some embodiments, the areas of highest injuries within a tissue are identified as peaks or valleys of emission (e.g., highs or lows of IR emission). In some embodiments, the severity of the injury is evaluated by quantifying a peak or a valley (e.g., by calculating an area under the curve representing the peak or valley, for example by calculating the area under half the height of the peak or valley). However, it should be appreciated that any technique may be used to quantify different regions of emission intensity.

In some embodiments, different levels of emission are determined using a detector (e.g., an IR detector, for example a far-IR detector) that can detect and/or display small differences in the amount of IR energy emitted from an object (e.g., small differences in IR emission intensity). In some embodiments, a detector can detect difference in IR emission intensities that correlate with a temperature difference of 1° C., or less (e.g., 0.1° C., 0.01° C., or 0.001° C.). In some embodiments, a detector includes or is connected to a display that contains a palette (e.g., a color palette, a greyscale palette, an intensity palette, a heat map, or any combination thereof) that can distinguish IR emission intensities that correlate with a temperature difference of 1° C., or less (e.g., 0.1° C., 0.01° C., or 0.001° C.). In some embodiments, a detector includes a display (e.g., a high resolution display) that contains a palette (e.g., color or greyscale or a heat map or other palette) encompassing a temperature range of 0° C. to 50° C., 10° C. to 50° C., 10° C. to 40 ° C., 15° C. to 40° C. or 18° C. to 37° C.

In some embodiments, patterns of IR emission levels from an injured tissue (e.g., a wounded tissue or a burned tissue, for example burned skin) can be used to rapidly evaluate the severity of the wound or burn (and/or patterns of burn severity) soon after the injury occurs. This is much sooner than is currently possible. This allows for an appropriate treatment to be implemented much sooner, without needing to wait for visual signs of wound or burn severity to develop. This means that treatment can be more effective. It should be appreciated that injuries such as burns (but also other wounds, for example resulting from blunt force trauma) are often complex. An injury (e.g., a burn) can produce a range of tissue injury from regions of severe injury to regions of mild injury. An injury (e.g., a skin injury) often includes a complex pattern of regions of severe injury (e.g., deep tissue burns) surrounded by regions of lesser injury (e.g., mild burns or superficial burns). It is often difficult to determine visually which areas of an injury are more severely affected than others for several days (during which the injured tissue progressively dies and/or heals). However, methods described herein can be used to identify a pattern of tissue injury (e.g., identifying skin areas having different levels of wound or burn severity within a region of injured skin). Accordingly, methods described herein can be used to map an injury to identify portions that are more severely injured than others and that will require more intense treatment than others. In some embodiments, specific levels of IR emission in one or more regions of a wound or burn (e.g., detected as temperature levels, or other measures of the amount of IR energy coming from the injury, for example measured as energy per unit area, or other measures of IR emission) can be used to determine the severity of the wound or burn or region thereof (for example with regions of greater injury having lower surface temperature than region of lower injury in some cases). However, in some embodiments, the depth of a wound or burn can be assessed by determining the rate of change of IR emission levels across a wound or burn. In some embodiments, steep changes as a function of distance are indicative of shallow wounds or burns whereas shallow changes as a function of distance are indicative of deeper wounds or burns. In some embodiments, a point or area of low IR emission can be used to identify a center of a region of burned tissue being evaluated. In some embodiments, the point or area of a burn that has the lowest level of IR emission can be identified as the point or area that is the “coldest” in an infrared image of the burned tissue. In some embodiments, this represents the region that is the most burnt or damaged. It should be appreciated that the center of a burn can be approximately the geometrical center of a burned region of tissue (e.g., skin). However, in many instances burns do not have regular or symmetrical geometric shapes. For example, burns caused by fires or chemical exposure can have complex shapes and patterns. Nonetheless, a region of low IR emission (e.g., the region of the burned tissue having the lowest levels of IR emission, or a local minimum of infrared emission surrounded by areas having higher levels of IR emission) can be used as a point of origin for an analysis to determine the severity of the burn (e.g., the depth of the burn). In some embodiments, the region of lowest IR emission (or one or more local minima of IR emissions) can be used as a reference point to evaluate the amount of change in IR emission levels as a function of the distance from the reference point(s). In some embodiments, a gradual change in IR emission levels from a point of low emission is indicative of a deep burn, whereas a rapid change in IR emission levels as a function of distance from a point of low emission is indicative of a shallow burn. Accordingly, the slope of change in IR emission levels as a function of distance from a point of low IR emission can be used to evaluate the severity and/or depth of a burn or of a region of a burn surrounding a local low point. It should be appreciated that reference rates of change (e.g., slopes of IR emission curves as a function of distance from a point of low IR emission) can be determined for different burn severities (burn depth, degree of burn, etc.). It also should be appreciated that reference rates for different burn severities can be determined for different types of tissue (e.g., skin), for different types of injury (e.g., fire, chemical exposure, radiation, etc.), for different ages of individual (e.g., child, teen, young adult, middle-aged, 0-10 year olds, 10-20 year olds, 20-30 year olds, 30-40 year olds, 40-50 year olds, 50-60 year olds, 60-70 year olds, 70-80 year olds, 80-90 year olds, etc.), different subjects (humans, non-human mammals, birds, reptiles, etc.), different disease conditions, and for other parameters. These reference rates of change can be used to evaluate a rate of change in a tissue being evaluated (e.g., at the time of or soon after an injury). However, in some embodiments, different relative rates of change in IR emission levels as a function of distance across one or more burned regions can be used to determine the relative severity of the different burned regions in a subject without needing to compare them to one or more external (or predetermined) references. In some embodiments, both external and internal rates of change can be used to evaluate the burn severity of a subject (e.g., a burn patient). In some embodiments, the value of the difference between the highest level of IR emission intensity and lowest level of IR emission intensity within the injured tissue, or the number of different levels of IR emission intensity that can be detected, or the value of the difference between the highest or lowest level of IR emission intensity and a reference level of IR emission intensity corresponding to unburnt skin, or any other measure of IR emission levels, or any combination thereof, may be used to evaluate the extent or severity of a burn.

Accordingly, patterns of burn severity can be identified for the subject based on patterns of IR emission levels and these can be used to provide an early patient prognosis and/or an early treatment recommendation (e.g., within one or a few hours of the injury). In some embodiments, methods described herein are used to determine the size and shape of burnt tissue. In some embodiments, methods described herein are used to identify the most severely burnt regions within a burnt tissue.

It should be appreciated that other tissue injuries (e.g., wounds) can be evaluated using similar techniques, including for example obtaining and analyzing IR emissions from an injured tissue and identifying one or more local maxima or minima (e.g., peaks or valleys) of IR emission. In some embodiments, the rate of change of IR emission across an injured tissue (e.g., from a reference point, e.g., a center, of a local peak or valley) is used to evaluate the severity of the injury. In some embodiments, the size of the range of IR emission intensities within the injured tissue (e.g., the value of the difference between the highest and lowest level of IR emission intensity, or the number of different levels of IR emission that can be detected, etc., or any combination thereof) is used to determine the severity of the injury at different positions in an injury (e.g., at different positions on injured skin of a subject). In some embodiments, the size of a local peak or valley of IR emission is used to determine the severity of the injury at that position. In some embodiments, IR levels or patterns are used to determine the size and shape of an injury. In some embodiments, IR levels or patterns are used to identify the most severely injured regions within an injured tissue.

In some embodiments, a method comprises identifying an IR maximum or minimum (e.g., a peak or a valley of IR radiation) in a tissue. In some embodiments, this peak or valley represents a region of maximal tissue damage (e.g., burn or other wound) within an area of injury. In some embodiments, the severity of the injury can be evaluated by determining the properties of the peak or valley (e.g., size, for example by evaluating the area under half the peak or valley height, or the slope of the IR changes surrounding the peak or valley) or by patterns or profiles of IR radiation surrounding the peaks or valleys. It should be appreciated that in some embodiments, the most injured tissue portion may be the portion with the highest IR emission (e.g., highest apparent temperature or “hottest”). However, in other embodiments, the most injured tissue portion may be the portion with the lowest IR emission (e.g., lowest apparent temperature or “coldest”). The IR emission from the most injured tissue will depend, at least in part, on the emissivity of the most injured tissue, in addition to factors such as the impact of the injury on the amount of blood flow under the injured region. In the case of a burn, the most severely burned area can be detected as the “coldest” area (the area with the lowest IR emission). Accordingly, in some embodiments a method comprises a) determining the center (or one or more regions of higher damage) of a complex burn by identifying the regions that have the lowest levels of infrared emission (e.g., are the coldest), b) determining regions of healthy tissue surrounding the burn, or regions of lowest burn (healthiest burn regions) by identifying regions that have the highest levels of infrared emission (e.g., the warmest regions surrounding a burned tissue), and c) determining curves of infrared emission change (e.g., curves temperature change), wherein the properties of the curves are indicative of the burn severity (for example, steep curves are indicative of less damage or shallow burns, flat curves are indicative of severe damage or deep burns). In some embodiments, there can be rings of high IR emission surrounding a center of a burn. In this case, a region of healthy tissue (e.g., as described in b) above) can be identified as a region of tissue or skin that has a consistent IR emission that is similar to other tissue or skin that is known to be healthy, for example, uninjured tissue or skin on the body of the injured subject. This level may be lower than a ring of high IR emission immediately surrounding a burn.

In some embodiments, an analysis of IR profiles or patterns can be performed as a vector analysis wherein the vectors radiate from the areas of most damage and their properties are indicative of the degree of burn at any particular location within the burn. However, any suitable analytical technique (e.g., mathematical analysis, computer-based analysis, calculation, geometric comparison, or other assessment or evaluation) can be used to determine one or more levels of burn severity (or other wound severity) as a function of detected patterns of IR emission levels over the surface of a burn (or a portion of a burn, or a portion of skin suspected of being burned or otherwise injured). In some embodiments, an analysis can be automated (for example with one or more analytical steps performed using a processor on a computer).

In some embodiments, patterns of burn severity based on IR emission patterns can be evaluated immediately after an injury or as soon as a few hours after a burn. In some embodiments, the patterns of emissivity from a burn are more distinct within the first few hours after a burn than a few days later. Accordingly, in some embodiments a wound or burn can be evaluated by determining a pattern of IR emissions from the wounded or burnt tissue within a few hours (e.g., 1-3, 3-6, 6-12, 12-18, 18-24 hours) of tissue injury. For example, in some embodiments, a wound or burn can be evaluated by determining a pattern of IR emissions from the wounded or burnt tissue within 1 hour of tissue injury (e.g., immediately after the injury, or 1-5 minutes, or about 5 minutes, 5-15 minutes, 15-30 minutes, about 30 minutes, 30-45 minutes, 45-60 minutes, or about 1 hour after tissue injury). In some embodiments, a wound or burn can be assessed by, for example, analyzing the slope of change in levels of emissivity along one or more directions in at least part of the wound or burn (e.g., in one or more directions radiating out from a region of low or high IR emission). In some embodiments, vectors representing the rate of change of IR emissivity can be determined. In some embodiments, vectors originating in an area of high wound or burn are determined. In some embodiments, an area of high burn is identified as an area of low emission (e.g., an area that is shown as cold using an IR detection device that is calibrated for and/or provides a temperature readout). In some embodiments, the location of the most burnt tissue is identified as the region with the lowest emission levels (e.g., showing up as the coldest using an IR detection device that is calibrated for and/or provides a temperature readout.). It should be appreciated that any suitable infrared detection device can be used. In some embodiments, an infrared camera can be used. In some embodiments, an infrared detection device is calibrated to detect different temperatures. However, in some embodiments, an infrared detection device is calibrated to be sensitive to different levels of IR emissivity that can be affected by surface properties independent of temperature. Accordingly, an infrared detection device can be calibrated to detect contrasts between different surface properties (e.g., whether they are shiny or not, or other surface properties) that can be detected even if they have the same temperature. Accordingly, an infrared camera (e.g., a forward looking IR camera or detector) or other infrared detection device can be calibrated to be sensitive to surface contrasts between different tissue regions (e.g., calibrated or limited to detect particular IR ranges, for example far IR or subsets of wavelength ranges within the far IR). In some embodiments, a detector device include two or more detectors, for example each for a different type of IR (e.g., two or three of near, mid, or far IR). In some embodiments, a detector device includes a single detector that can detect two or more types of IR (e.g., two or three of near, mid, or far IR).

It should be appreciated that different IR spectra can be evaluated. In some embodiments, near IR is evaluated. In some embodiments, mid-IR is evaluated. In some embodiments, far IR is evaluated. In some embodiments, two or more are evaluated. In some embodiments, IR patterns are evaluated and or overlaid with other information (e.g., visible or UV patterns or images). In some embodiments, mid-IR data can be detected by using a suitable detector and a suitable excitation energy. If two or more detectors are used, they can be aligned to allow the images or data to be overlaid.

It should be appreciated that a rapid and correct assessment of a tissue burn can be clinically important. As a general clinical rule, an adult with burns on more than 20% of the body surface area, or a child with burns on more than 10% of the body surface area, will require intravenous fluid replacement. However an intravenous line may be necessary to achieve adequate analgesia for a much smaller burn, and in some instances (e.g., for some children) fluid replacement may be required because of vomiting. It also should be appreciated that a patient's (e.g., a human patient's) prognosis can be evaluated based upon the percentage of the patient's body surface area that is burned. In some embodiments, as a rough guide, if the age of the patient and percentage of body burned are added together, a score of 100 or above is indicative that the burn is likely to be fatal. For example, a child can survive a relatively large burn, but a smaller burn is potentially fatal for an elderly patient.

Similarly, for other injuries, an early assessment of an injury can be helpful to initiate an appropriate and/or targeted treatment as soon as possible.

In some embodiments, IR imaging as described herein also can be used to evaluate or detect other conditions such as diseases (e.g., skin lesions, cancerous or pre-cancerous lesions, acne, rashes, psoriasis, other inflammatory conditions, or other skin conditions). As described herein, an IR pattern or profile can be used to evaluate and or determine the extent and or severity of a condition (e.g., a skin condition). In some embodiments, a method involves comparing IR patterns or profiles to reference patterns or profiles associated with a condition of interest. In some embodiments, an IR pattern can be used as the basis of a targeted treatment. For example, treatment can be targeted to those areas that are identified as being the most severely affected. In some embodiments, a bandage (e.g., bandaid or other medically suitable support material, for example with or without an adhesive strip for application to a tissue surface such as a skin surface) can be prepared with a spatial pattern of treatment (e.g., of one or more antibiotics, anti-inflammatory compounds, or other treatment materials) that corresponds to one or more features of the IR pattern. In some embodiments, a bandage is a therapeutic bandaid. In some embodiments, a bandage is a preparative bandaid. For example, it can contain a pattern of debriding compositions to prepare a burned or otherwise injured tissue for subsequent treatment. In some embodiments, a bandage may contain both preparative and treatment compositions. In some embodiments, a bandage can be printed with an appropriate pattern of one or more compositions. However, compositions can be deposited on a bandage in an appropriate pattern using any suitable technique. It should be appreciated that a bandage can be of any suitable size to cover an area of injured (e.g., burned or wounded) tissue. In some embodiments, two or more bandages may be used to cover an injured tissue area or a portion thereof.

It should be appreciated that infrared contrast imaging as described herein can be used to evaluate different aspects of biological tissues, and also can be used to evaluate other materials (for example metals, wood, plastic, etc., for example to detect damage or other physical modifications).

These and other aspects are described in more detail below and with reference to the drawings.

### DESCRIPTION OF THE DRAWINGS

FIG. 1A illustrates a technique for obtaining IR data for different types of burns;

FIG. 1B illustrates Jackson's Burn Model showing distinct areas of tissue damage within a burn;

FIG. 1C shows infrared (IR) emission from a burn and illustrates how a burn can be assessed using IR-based techniques described herein;

FIG. 2A illustrates a burn model using pig skin showing surface squares of tissue regions that are targeted for burning, different squares can be exposed to different temperatures and/or for different times to model different levels of burn severity;

FIGS. 2B and 2C illustrate IR spectra from burned tissue regions within the first day after a burn injury;

FIG. 2D illustrates a non-limiting technique for analyzing IR data;

FIG. 3A illustrates a burn model;

FIG. 3B shows levels of IR emissions across 3 different burned regions;

FIG. 3C illustrates deep burn patterns (green and blue) and shallow burn patterns (red);

FIG. 4A shows IR emission patterns from non-limiting examples of burned tissue;

FIG. 4B illustrates a model of a deep burn;

FIG. 4C illustrates a model of a shallow burn;

FIG. 5 shows a non-limiting example with several (7) distinguishable layers of tissue disruption one day after a burn;

FIG. 6A shows the IR patterns from top views of different burns;

FIGS. 6B and 6C show depth profile models;

FIGS. 7A-7B show the data and models of FIGS. 6A-6C in more detail;

FIG. 8 shows different IR profiles corresponding to different lines across one or more burned tissue regions;

FIG. 9A shows IR patterns of scars at 28 days after injury;

FIG. 9B shows IR patterns of burned tissue at 24 hours after injury;

FIG. 9C illustrates IR patterns of burned tissue at 24 hours, 72 hours, and 7 days after injury;

FIG. 9D illustrates that IR data at 24 hours after injury correlates with wound contraction and scar depth 4 weeks after injury;

FIG. 9E illustrates that IR data at different times after injury correlates with scar depth 4 weeks after injury;

FIG. 10 shows IR patterns for scars at 672 hours after injury, lines 4 and 5 show minimal scaring, characteristic ring destruction patterns are still evident on scarred skin whereas minimally scarred tissue shows no ring destruction;

FIG. 11 shows a high definition IR image of a day old burn;

FIGS. 12A-B provide a model for real burns based on infrared contrast imaging (IRCI) data, illustrating a shallower pattern of IR changes radiating from the center of a deep burn than for a shallow burn;

FIGS. 13A-C provide an example of an analysis of a burned tissue model and illustrates how shallow and deep burns can be identified based on patterns of IR emission (and the extent of change in IR emission from the center of a burn;

FIG. 14 illustrates vectors representing patterns of IR emission changes from the centers of burned regions that can be used to evaluate burn depth (and, for example, to distinguish shallow burns from deep burns);

FIG. 15 illustrates non-limiting examples of IR emission profiles (shown as temperature profiles) across the diameter of a burn;

FIG. 16 illustrates non-limiting examples of vectors radiating from the points of lowest emission for shallow and deep burns;

FIG. 17A illustrates further non-limiting examples of vectors radiating from the points of lowest emission for several shallow and deep burns;

FIG. 17B illustrates a non-limiting example of a profile obtained from an IR image containing an irregular burn pattern;

FIG. 18A illustrates an IR pattern of a skin condition;

FIG. 18B illustrates several bandages printed with preparation or treatment compositions deposited in a pattern corresponding to one or more features of the IR pattern of the skin condition; and

FIG. 18C illustrates a bandage of FIG. 18B applied to the skin condition of FIG. 18A.

### DETAILED DESCRIPTION OF CERTAIN EMBODIMENTS OF THE INVENTION

In some embodiments, aspects of the invention relate to methods and devices for evaluating tissue injuries by analyzing emission spectra and/or emission levels (e.g., near IR, mid IR, far IR, or a combination thereof) from injured tissue. In some embodiments, aspects of the invention relate to methods and devices for detecting or evaluating tissue conditions (e.g., diseases or disorders) by analyzing emission spectra and/or emission levels (e.g., near IR, mid IR, far IR, or a combination thereof) from a target tissue (e.g., skin) suspected of having a disease or disorder. In some embodiments, infrared emissions from a tissue region can be used to evaluate the presence and/or extent of an injury or condition of the tissue region. According to aspects of the invention, the level of infrared emission from a tissue is affected by physical changes in the tissue that can be caused by injury or condition. In some embodiments, an injury is a burn. In some embodiments, an injury is a traumatic injury. In some embodiments, an injury is a cut, a tear, an abrasion, or other physical disruption of tissue integrity. In some embodiments, an injury can result from a pressure wave (e.g., from an explosion or other source of pressure blast). In some embodiments, a tissue condition can be a skin disease or disorder (e.g., an infection, inflammation, psoriasis, acne, cancer, or other condition or disorder).

As used herein, the term “level of infrared emission” or “infrared emission level” refers to an amount of infrared energy emitted from a volume or surface. . In some embodiments, a level of infrared emission is a mean power per unit area (intensity) of radiation emitted from a volume or surface (e.g., a tissue or skin surface) across a particular band of wavelengths in the infrared range. In some embodiments, a level of infrared emission is a time averaged intensity of radiation emitted from a volume or surface (e.g., a tissue or skin surface) across a particular band of wavelengths in the infrared range. In some embodiments, a level of infrared emission is a maximum intensity of radiation emitted from a volume or surface (e.g., a tissue or skin surface) across a particular band of wavelengths in the infrared range over a period of time. In some embodiments, a level of infrared emission is a root mean square of intensity of radiation emitted from a volume or surface (e.g., a tissue or skin surface) across a particular band of wavelengths in the infrared range over a period of time.

In some embodiments, a level of infrared radiation is determined that comprises power per unit area (intensity) of radiation emitted, transmitted or reflected from a volume or surface (e.g., a tissue or skin surface) across a particular band of wavelengths in the infrared range. In some embodiments, levels of infrared radiation reflect relative intensities of infrared radiation emitted, transmitted, and/or reflected from across a volume or surface.

In some embodiments, infrared radiation from an injured tissue or object primarily comprises emitted infrared radiation and there is very little transmitted or reflected infrared radiation. In some embodiments, reflected infrared radiation may be reduced by using a filter or polarizing screen above or around an injured area to prevent external infrared radiation from reaching the tissue.

In some embodiments, levels of infrared radiation are determined using an infrared detection system (e.g., spectroscopy, thermography) to measure infrared radiation emitted (as opposed to being transmitted or reflected) from a volume or surface. However, in some embodiments, levels of infrared radiation are determined using infrared detection system (e.g., spectroscopy, thermography) to measure infrared radiation emitted, transmitted and/or reflected from a volume or surface.

In some embodiments, infrared radiation can be used to determine the temperature of objects where the emissivity of the surface is known or estimated. In some embodiments, levels of infrared radiation are displayed or reported as temperatures, but the differences in apparent temperature can be indicative of differences in emissivity (e.g., caused by a tissue injury or disorder) in addition to or instead of actual differences in temperature (e.g., due to inflammation or differences in blood flow through an injured tissue). It has been appreciated, as described herein, that tissue changes caused by an injury such as a burn can change the level and/or pattern of IR radiation that is emitted from the injured tissue, and that the degree of injury affects the amount of IR radiation that is emitted. The change in the amount of IR radiation that is emitted is affected in part by immediate changes to the tissue upon injury that alter the emissivity of the tissue (e.g., changes in density, surface texture, water content, etc., that are impacted by the injury). Accordingly, the amount of IR radiation that is emitted from an injured tissue (e.g., wounded or burned skin) can be used to evaluate the severity of the injury soon after the time of injury (e.g., within minutes to hours of the injury) as opposed to waiting for visual signs of injury to develop through natural physiological healing processes (that can take several days). This allows for appropriate medical treatment to be applied early after injury and also for the treatment to be appropriately targeted (e.g., to areas of most severe injury). Similarly, the extent and or severity of a disease or disorder that affects skin or tissue properties can be detected and/or evaluated using an IR analysis as described herein.

In some embodiments, the amount of IR radiation that is emitted from an injured tissue is measured using a device (e.g., a camera) that provides (e.g., displays) apparent temperatures of the tissue. However, other devices and techniques for detecting, measuring, or displaying the amount of IR radiation also can be used as aspects of the invention are not limited in this respect. In some embodiments, absolute levels of radiation do not need to be calculated, determined, and/or displayed, because differences in tissue injury severity or tissue condition can be determined based only on relative amounts of IR radiation for different portions of the tissue (e.g., of the injured tissue).

In some embodiments, an infrared camera or other infrared detection device can be used to detect or measure infrared emission levels from a target tissue of interest (e.g., from the surface, e.g., skin, of a tissue region that is injured or suspected to be injured and/or including surrounding tissue) of a subject. In some embodiments, far infrared emission levels are detected. However, in some embodiments, near or mid-infrared emission levels are detected, as aspects of the invention are not limited in this respect. In some embodiments, data obtained from an infrared detection device (e.g., an infrared camera) can be analyzed on a computer to detect patterns and/or levels of infrared emissions associated with the presence and/or extent of injury. These levels can be displayed (e.g., on a screen of the computer) using any suitable scheme (e.g., different colors, different identifiers for different zones, or any other suitable scheme, or any combination thereof). In some embodiments, the infrared emission information can be analyzed using a processor that is incorporated in the infrared detection device and the detected levels of infrared emission and/or the correlated levels of tissue injury can be displayed directly on the infrared detection device (e.g., camera). For example, different levels of hematomas, different degrees of burn, different burn depths, or other different levels of tissue damage can be displayed (e.g., on a screen) on the detection device. In some embodiments, the infrared detection device can be a hand-held camera (e.g., a forward looking infrared camera). However, immobilized detection devices (e.g., an immobilized camera, for example on a support such as a tripod) or larger detection devices also can be used as aspects of the invention are not limited in this respect. In some embodiments, a device can detect only one type of IR radiation (e.g., near, mid, or far). However, in some embodiments, a device can detect two or more types of IR radiation. In some embodiments, two or more different devices can be used. Information corresponding to different types of IR radiation can be overlaid and or combined with other information including for example visual or UV information.

In some embodiments, detection devices can be head-worn (WYSIWYG). In a non-limiting example of a head worn device, one or both eyes can see IR wavelengths, visible wavelengths, and or UV wavelengths. In some embodiments, both eyes can see one or more different wavelengths (or wavelength ranges) allowing for a stereoscopic display. In some embodiments, one or more images can be displayed as overlays to provide composite views of different types of data.

In some embodiments, aspects of the invention relate to using infrared patterns to evaluate tissue injuries (e.g., a wound or burn severity) or conditions based on the surface infrared emission levels (for example from skin) without requiring any dyes, labels, or energy input. In some embodiments, no irradiation is required since techniques described herein do not rely on reflected IR. For example, far IR data corresponding to thermal radiation can be obtained without using a dye or excitation energy. However, in some embodiments, one or more dyes or excitation energies can be used. For example, mid IR data can be obtained using an appropriate excitation energy in addition to a mid IR detector.

In some embodiments, the infrared emissions utilized with the methods and devices disclosed herein have a wavelength in the range of 0.7 μm to 25 μm or 8 μm to 25 μm. In some embodiments, the infrared emissions comprise emissions in the near infrared range, e.g., emissions having a wavelength of about 0.7 μm to about 1.0 μm. In some embodiments, the infrared emissions comprise emissions in the short-wave infrared range, e.g., emissions having a wavelength of about 1 μm to about 3 μm. In some embodiments, the infrared emissions comprise emissions in the mid-wave infrared range, e.g., emissions having a wavelength of about 3 μm to about 5 μm. In some embodiments, the infrared emissions comprise emissions in the long-wave infrared range, e.g., emissions having a wavelength of about 8 μm to about 12 μm, or about 7 μm to about 14 μm. In some embodiments, the infrared emissions comprise emissions in the very-long wave infrared range, e.g., emissions having a wavelength of about 12 pm to about 30 μm. In some embodiments, the infrared emissions comprise emissions in the far infrared, e.g., emissions having a wavelength of about 15 μm to about 1000 μm.

The use of IR patterns to evaluate the severity of an injury (e.g., a skin wound or a skin burn) is illustrated by experimental models described with reference to some of the figures. However, it should be appreciated that these experiments are non-limiting and the techniques can be used to evaluate injuries (e.g., wounds or burns) in other contexts (e.g., for human tissue) or other conditions (e.g., skin diseases or disorders).

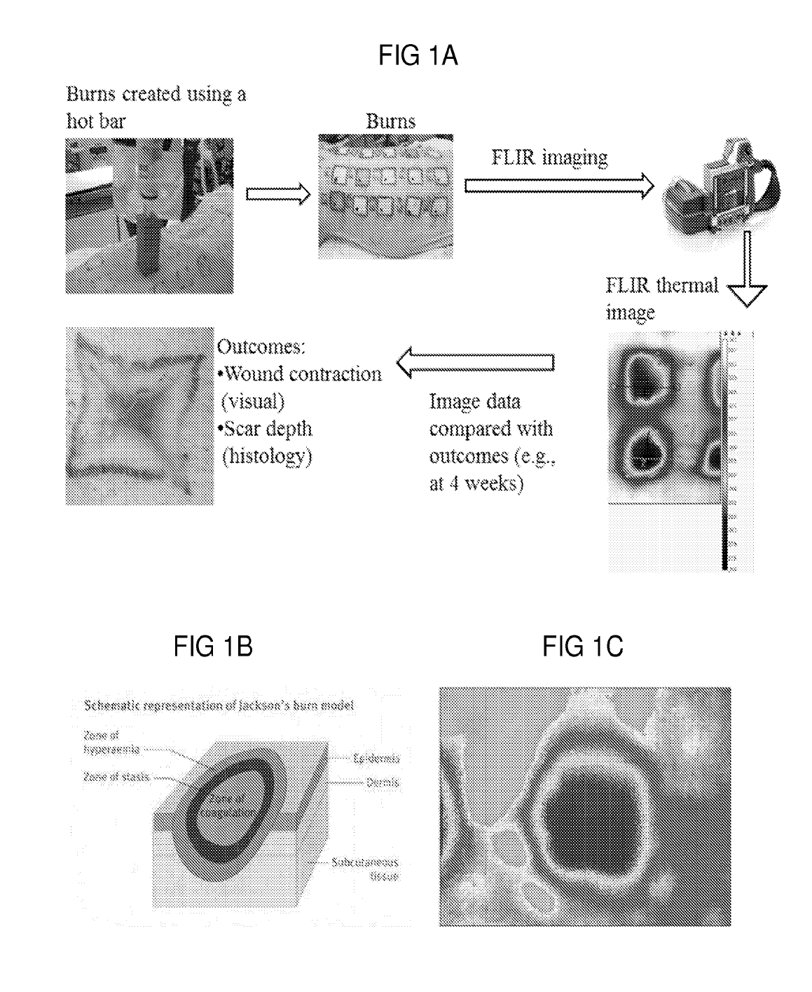

FIG. 1A illustrates a non-limiting method for obtaining IR data for different burns and correlating the data with different outcomes. Burns are created by contacting one end of heated metal bar (e.g., an aluminum bar) to the skin of a test animal (e.g., a pig). The metal bar can be heated using any suitable technique (e.g., by maintaining it in a water bath at a set temperature). Different temperatures and different contact times can be used to generate different levels of burn. For example, contact with a bar at 70° C. for 20 seconds can be used to create a superficial dermal burn, contact with a bar at 80° C. for 20 seconds can be used to create a mid-dermal burn, and contact with a bar at 80° C. for 30 seconds can be used to create a deep dermal burn. IR emissions from the burns can then be detected and/or measured using an appropriate IR detector. For example, the burns can be photographed using an IR camera (e.g., a forward looking infrared camera, for example, a FLIR T300 available from FLIR Systems, Inc.). The IR data (e.g., the thermal image obtained with the IR camera) can then be analyzed and compared with the outcomes of the burns (e.g., the wound contraction and/or the scar depth) at a later time (e.g., 4 weeks after the burn). It should be appreciated that burn outcomes can be measured using any suitable method. For example, wound contraction can be evaluated visually, and scar depth can be evaluated histologically. However, other techniques can be used.

Different burn models exist. FIG. 1B illustrates a general burn model (Jackson's Burn Model) showing distinct areas of tissue damage within a burn. In FIG. 1B, the central zone of coagulation represents the zone of severe damage caused by primary injury. In general, these tissues do not recover and will eventually slough off as dead tissue over time. Surrounding the central zone of coagulation lies the zone of stasis. This zone is characterized by less damaged tissue in which inflammation occurs and vascularity is impaired. Tissue in this zone has the potential to recover under correct conditions. The outer layer is the zone of hyperaemia characterized by tissue with intense vasodilatation and increased blood flow. The margins between the zones are not static since they are influenced by local and systemic factors, including reduced blood flow. In some embodiments, excessive edema tends to extend the zone of coagulation and hence the area of tissue necrosis. Under favorable conditions, the margin of the central zone remains static and the zone of stasis shrinks as it is replaced by the zone of hyperemia. According to aspects of the invention, different burn zones can be identified by analyzing IR data from a burned tissue as described herein.

It should be appreciated that a burn wound is dynamic and subject to the effect of secondary injury. For example, the burn may deepen if the blood supply of the wound is impaired (e.g., due to hypovolaemia, hypotension, or if infection occurs).

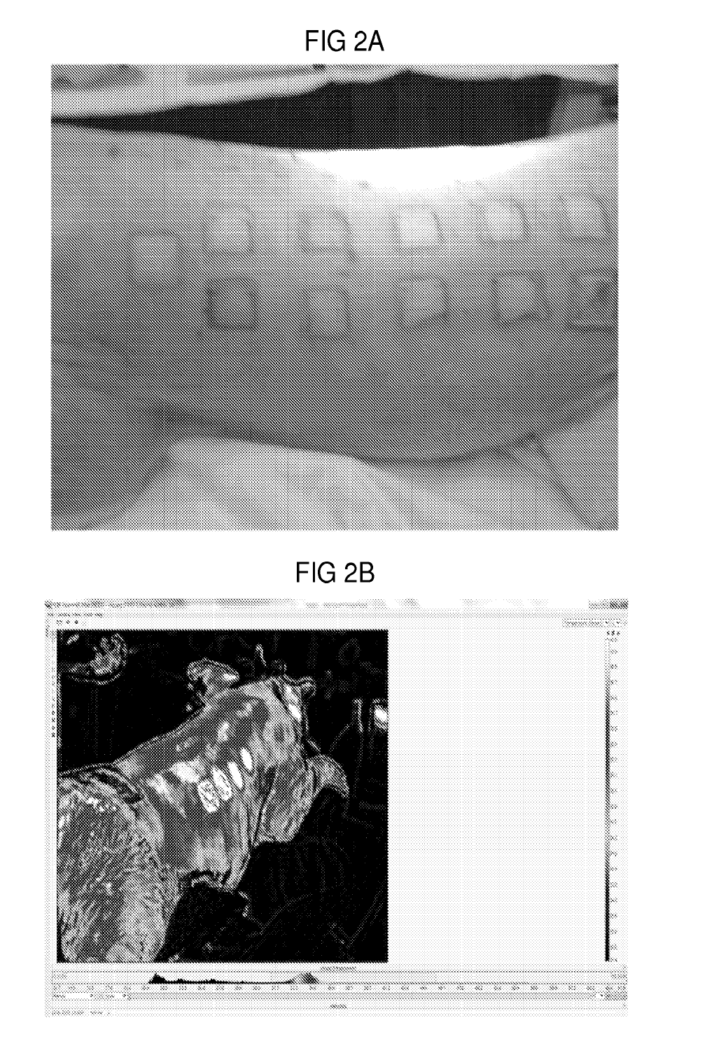

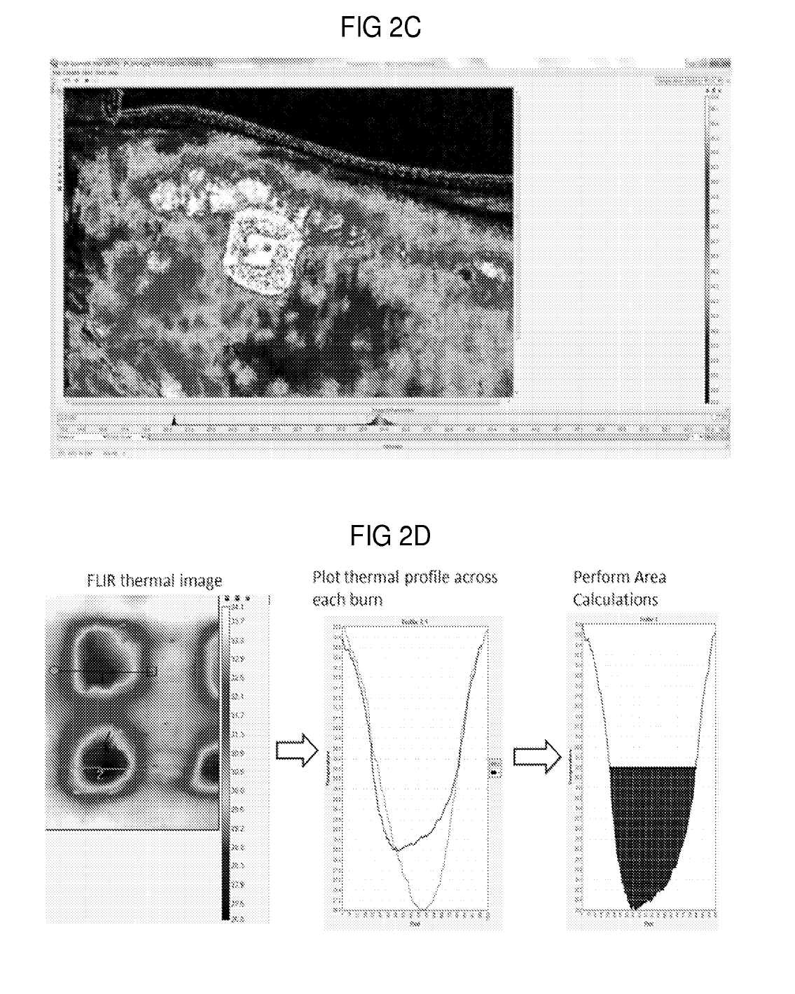

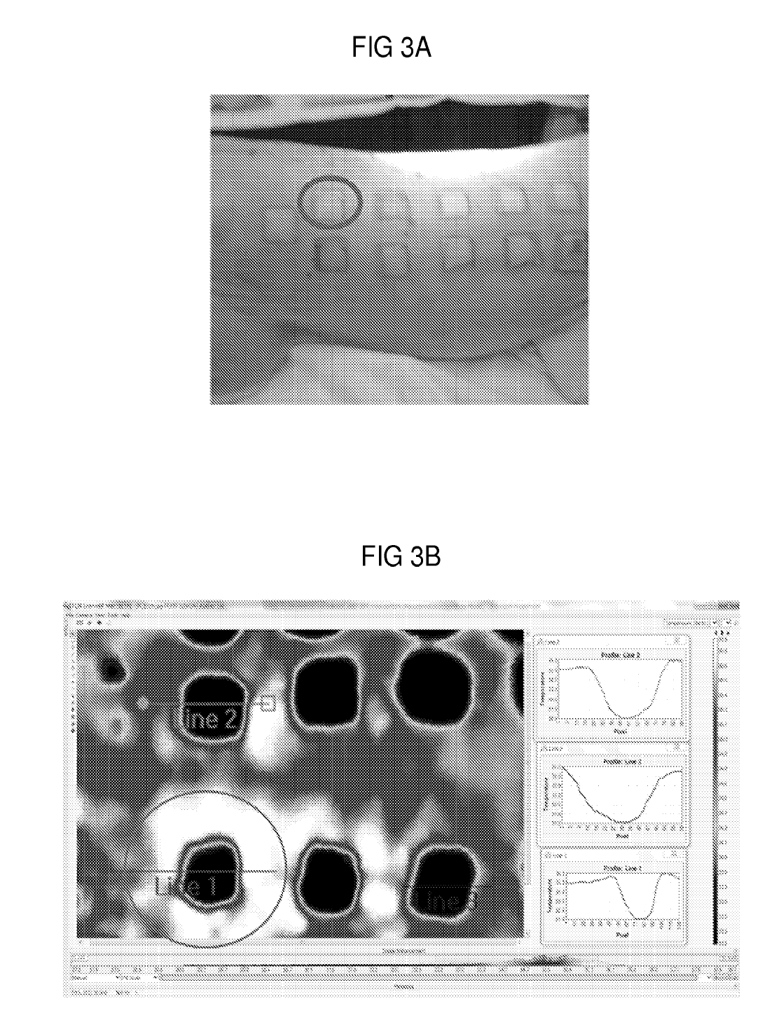

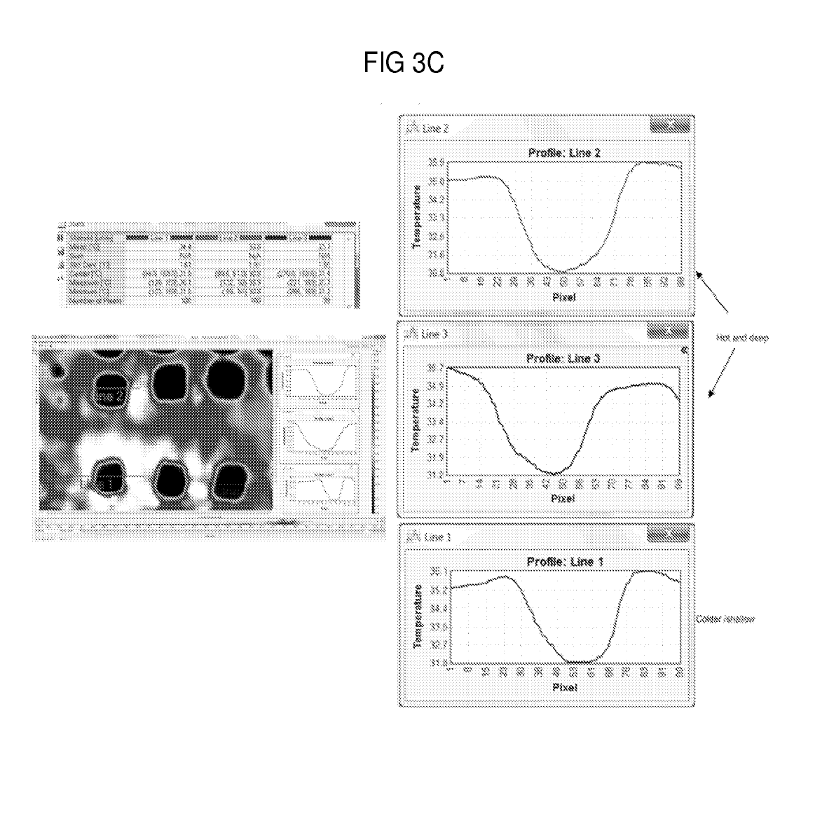

FIGS. 2A and 3A illustrate a non-limiting examples of a burn model using pig skin, where different regions (e.g., different squares) are exposed to different temperatures for different lengths of time to provide models for different levels of burn severity. FIG. 2B shows IR emission images of the burned tissue. FIG. 2D depicts a method for analyzing IR image data and illustrates that, in some embodiments, a mathematical size or quantification of a peak or valley (e.g., area under curve) can be used to assess the extent and/or severity of a burn or wound. In some embodiments, a larger area may be the result of colder temperatures, which may be due to lower perfusion and/or indicative of greater tissue damage

FIGS. 3B-3C show an analysis of the IR emission across several burned regions. It should be appreciated that the IR image in FIG. 3B is a different view from the visual picture of FIG. 3A. The spot location is the same as circled, but the visual picture is shown with the spine in the middle. Two different sets of data are shown at the same time. This may be more similar to an actual burned area where the degrees of burn are intermixed. FIG. 3B shows an example of IR data for day one after a burn. The data is shown as a temperature profile, but it should be appreciated that data can be evaluated and/or displayed as an IR emission profile and does not need to be converted into temperature. In this non-limiting example, baseline skin temperature in the burn area is 34 to 34.4° C., temperature in the burn is about 33.2° C., and temperature in the areas surrounding the burns is 36.1 to 36.9° C. A histological analysis of these areas can be used to evaluate the extent of damage below the surface of the burned regions so that IR profiles for different levels of burn severity can be confirmed.

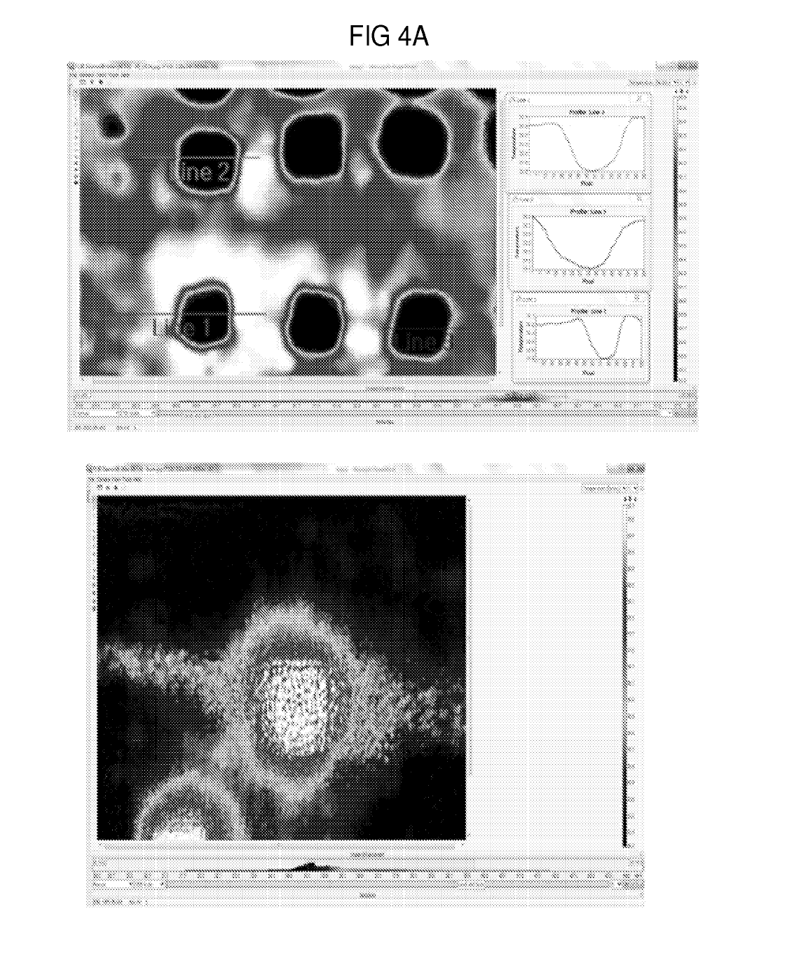

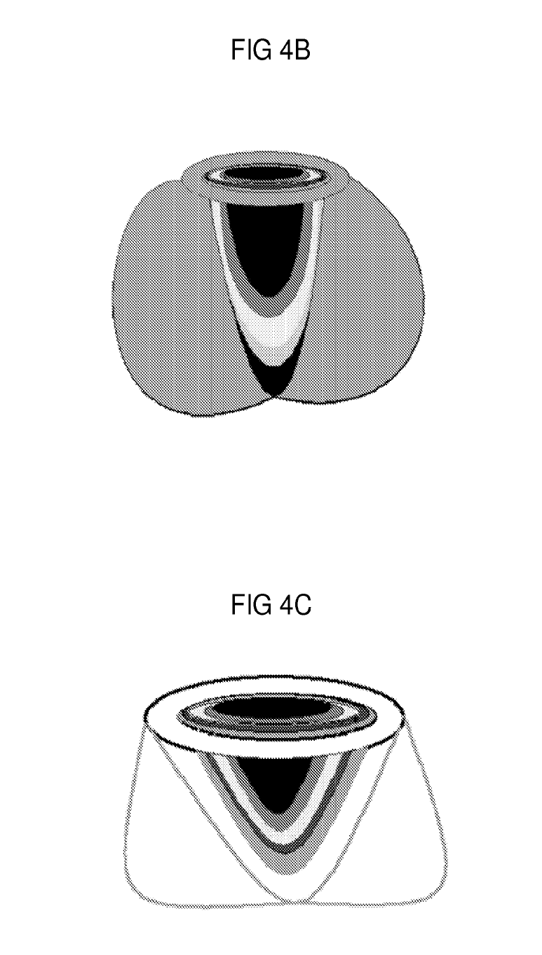

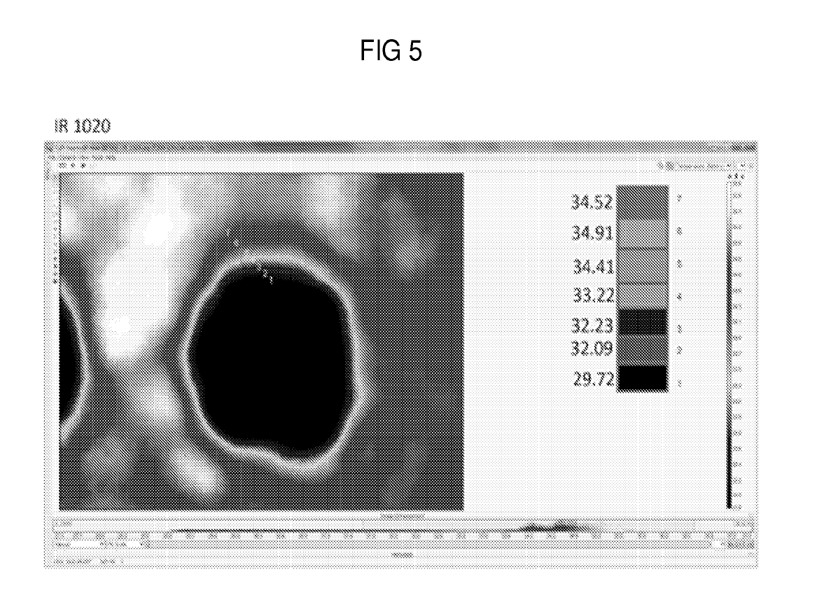

FIG. 4 shows IR data in more detail and illustrates models of a deep burn (see FIG. 4B) and a shallow burn (see FIG. 4C). It should be appreciated that deep burns may have low vascular destruction deeper down, in some embodiments. This may be reflected by a low central temperature within a few hours after a burn. In some embodiments, a difference in emission levels between different regions can be highlighted if there is no, or less, tissue healing or inflammation that can make the data more difficult to evaluate.

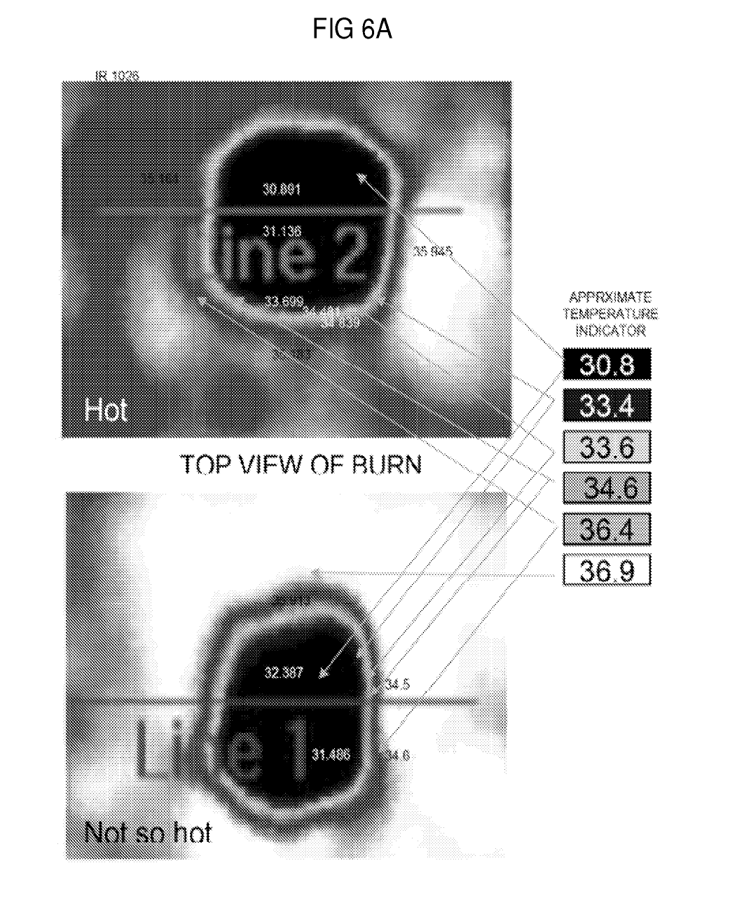

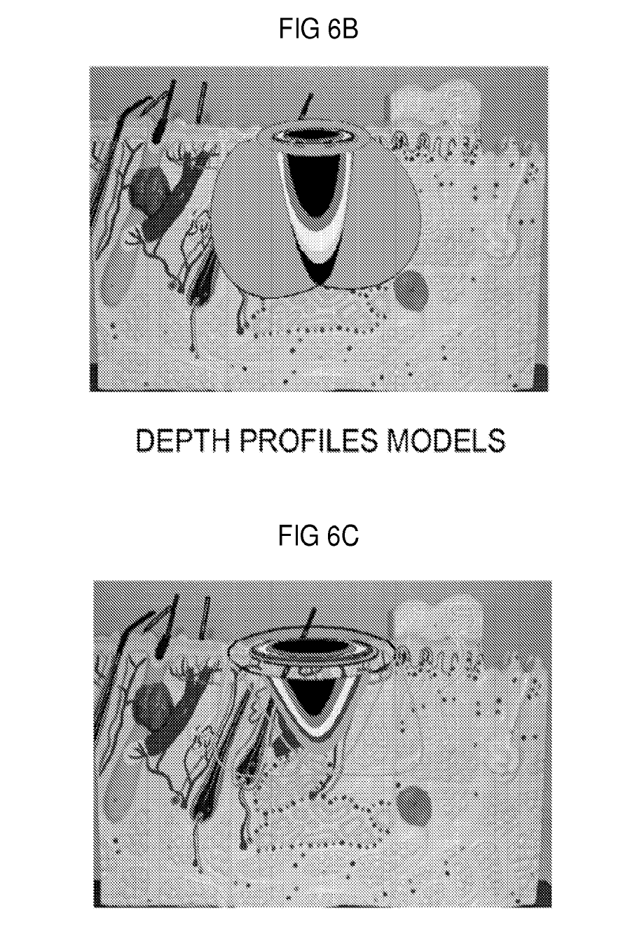

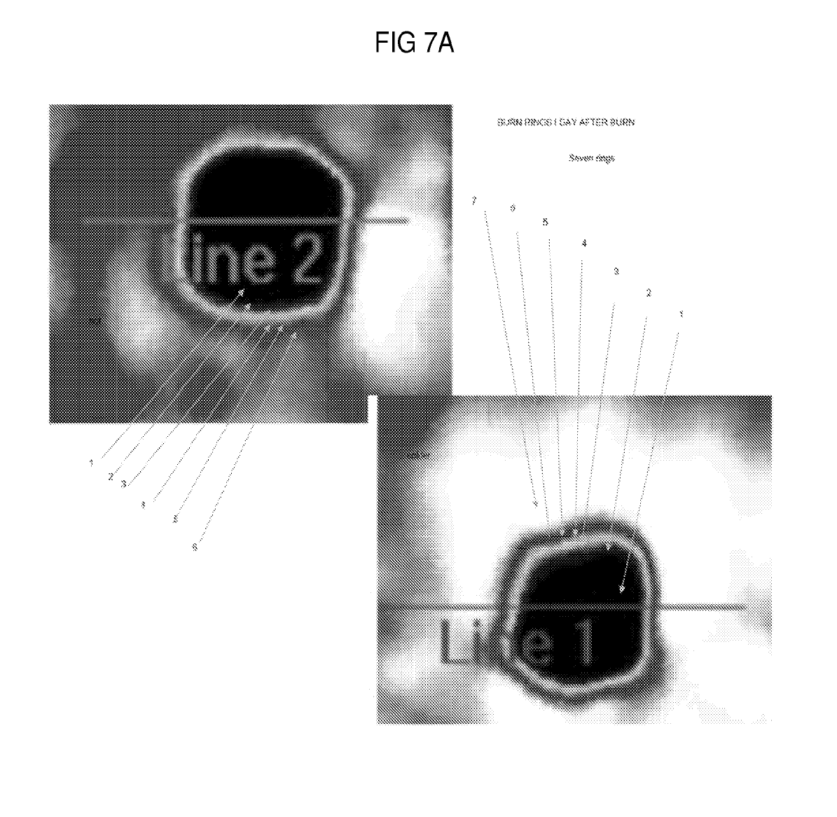

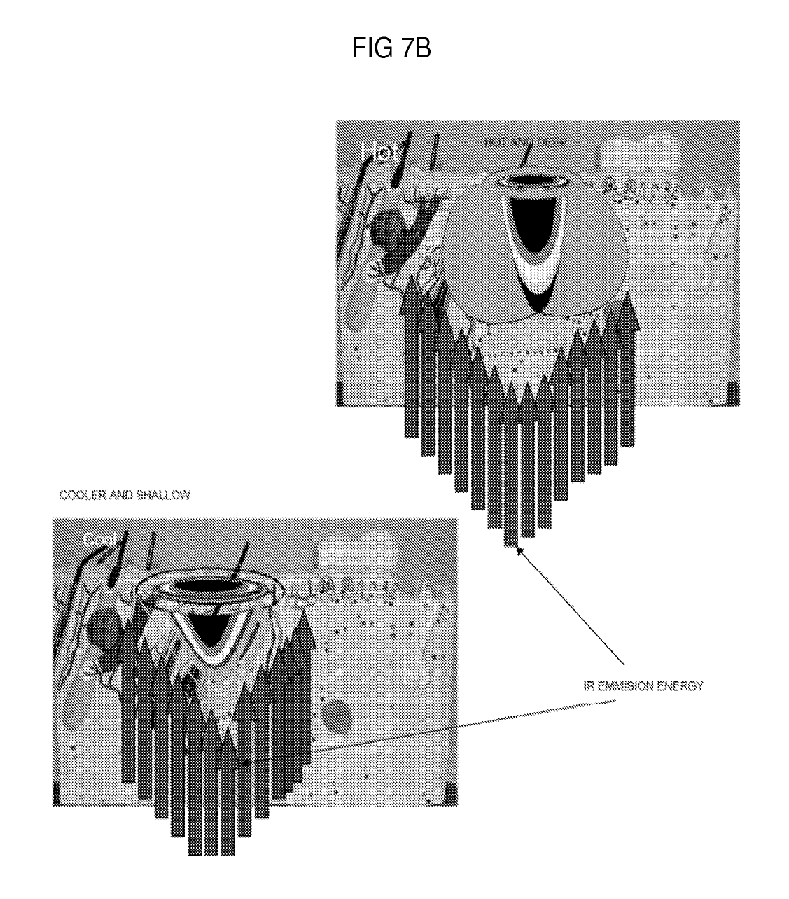

FIG. 5 shows a non-limiting example with several (7) distinguishable layers of tissue disruption one day after a burn as detected by different levels if IR emission. It should be appreciated that different levels of IR emission can be distinguished based on the calibration of the IR detector. FIGS. 6 and 7 show non-limiting examples of IR top-view patterns and models of different burn depth profiles. In some embodiments, the outer perimeter is cooler in the burns resulting from hotter and/or longer burning conditions relative to the outer perimeter of the burns resulting from cooler and/or shorter burning conditions. In some embodiments, IR energy comes through the tissue, but because the hotter and deeper burning conditions produce more damage and deeper damage (loss of blood flow, loss of temperature, dehydration, small inflammatory response) that may not be evident on the surface, the IR energies are absorbed differently than for shallower burns (e.g., resulting from exposure to shorter burning conditions). Accordingly, shorter and shallower burns appear overall hotter (higher IR emissions) than the deeper burns. In some embodiments, shallower burns are characterized by having a higher number of readily distinguishable levels of IR emission (e.g., a higher number of different heat levels) than deeper burns. For example, in some embodiments shallow burns have seven different layers radiating from the burn center, whereas deep burns only have six. In some embodiments, the colder outside perimeter is cooler for the deep burns than for the short and shallow burns. In some embodiments, shallow burns allow higher IR emissions (e.g., detected as temperature using an IR detector calibrated to detect temperature) to radiate from underneath the burn and be detectable at the surface through the wound.

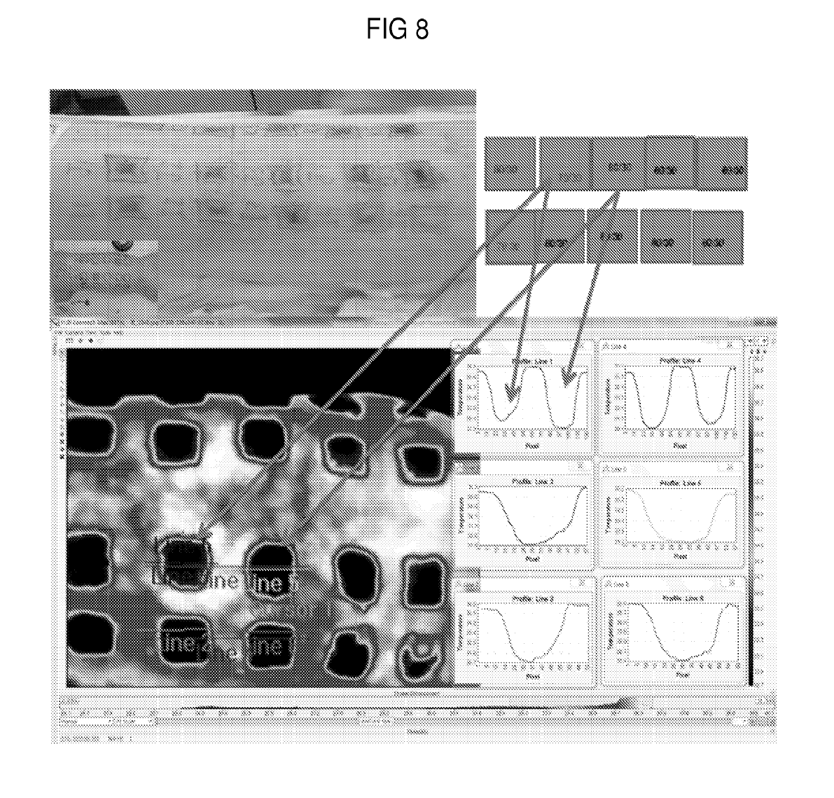

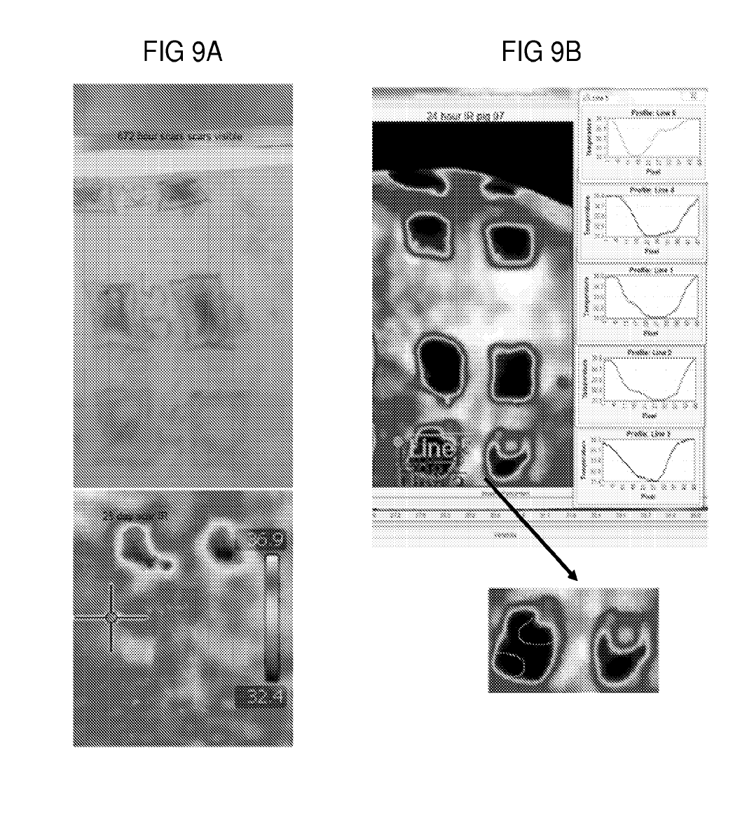

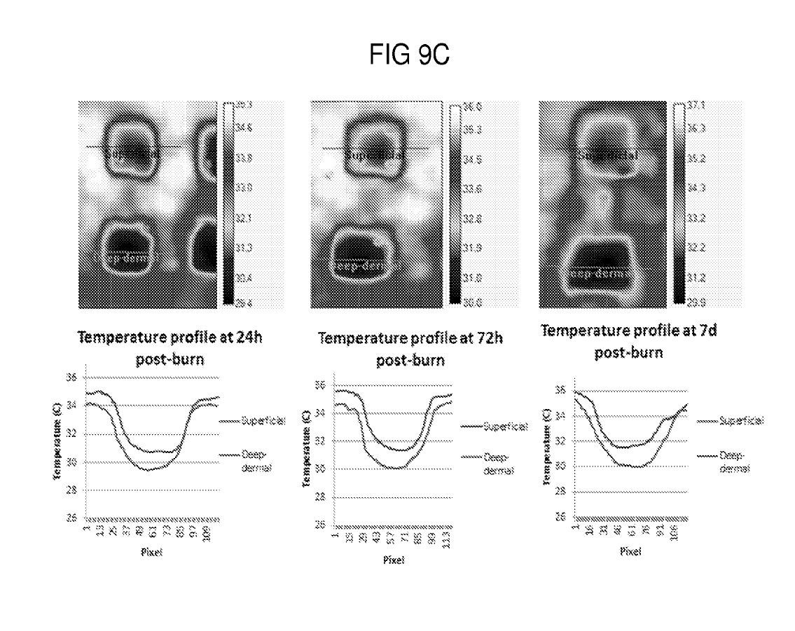

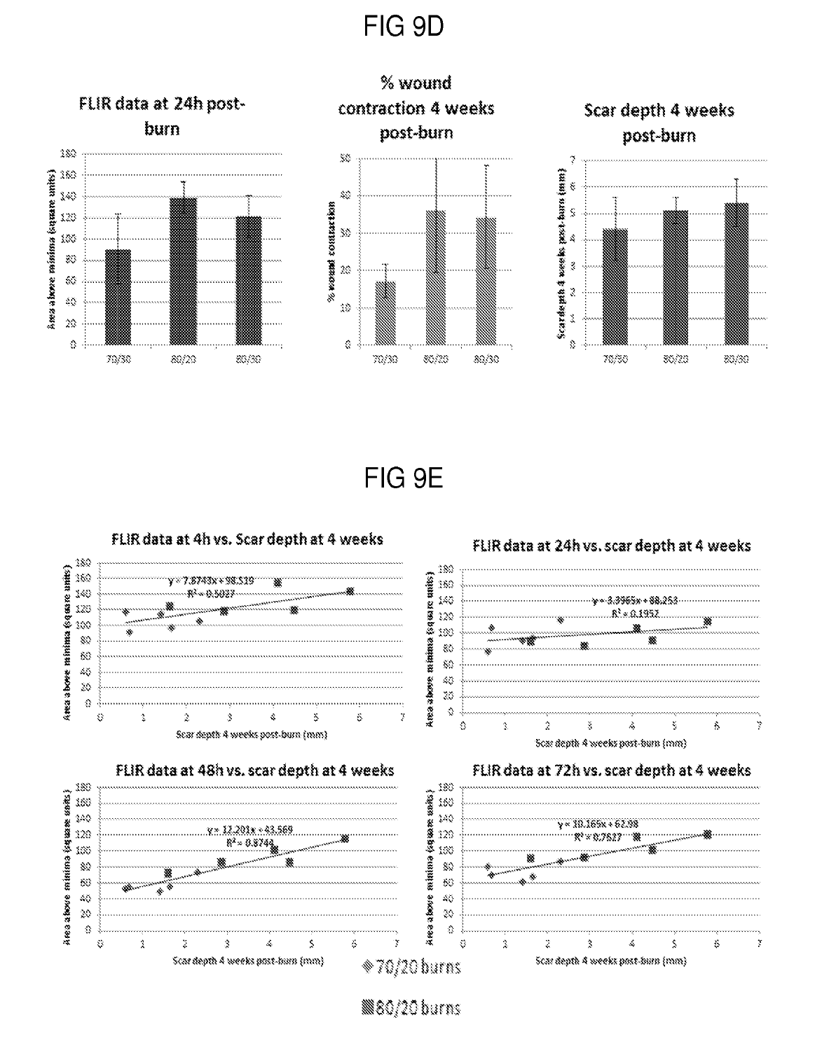

FIGS. 8-10 show different patterns of IR profiles and scars for different burns at different times after injury FIG. 9A shows IR patterns of scars at 28 days after injury. FIG. 9B shows IR patterns of burned tissue at 24 hours after injury. FIG. 9C illustrates IR patterns of burned tissue at 24 hours, 72 hours, and 7 days after injury. A temperature profile is shown for both a superficial and a deep-dermal burn. The shape of the profile is similar for both superficial and deep-dermal burns, but the detected temperatures are lower for the deep dermal burns. The burn profiles are detectable at both early and later time points, although there is a detectable shift in the profiles towards higher temperatures at later times after the injury. FIG. 9D illustrates that IR data at 24 hours after injury correlates with wound contraction and scar depth 4 weeks after injury. The first panel shows IR data 24 hours after a burns of three different depths. The second panel shows the % wound contraction for these three burn types 4 weeks after the injury, and the third panel shows the scar depths for the three burn types 4 weeks after the injury. The scar depth is evaluated by histology at 4 weeks. The IR data is consistent with the physiological outcome. For example, the 70/20 burn (70° C. for 20 seconds) has a lower depth than the 80/30 burn (80° C. for 30 seconds). FIG. 9E illustrates that IR data at different times after injury correlates with scar depth 4 weeks after injury.

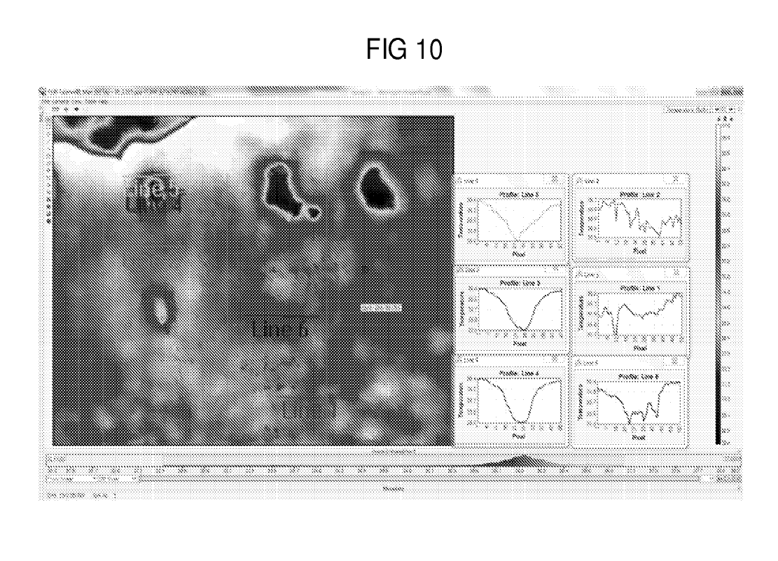

In FIGS. 10, 14 and 15 show minimal scarring after 672 hours. However, characteristic ring destruction pattern is still evident on scarred skin, wherein minimally-scarred tissue shows little or no ring destruction.

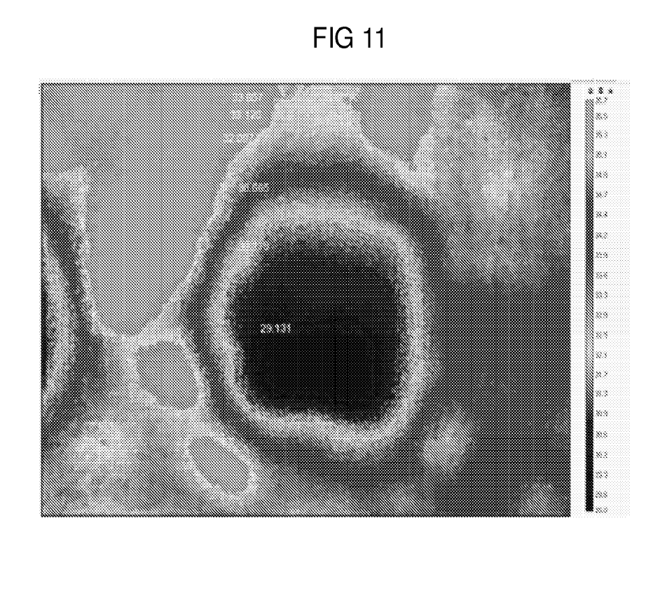

FIG. 11 shows a high definition IR image of a day old burn. In some embodiments, in a non-scarring burn the temperature in the center is warmer than for a scarring burn, and the temperature change from the center to the outside of a non-scarring burn goes up towards the outer edge. In contrast, a scarring burn does not change temperature as much. Also, scarring burns are typically colder than an average skin temperature (even at 672 hours after a burn).

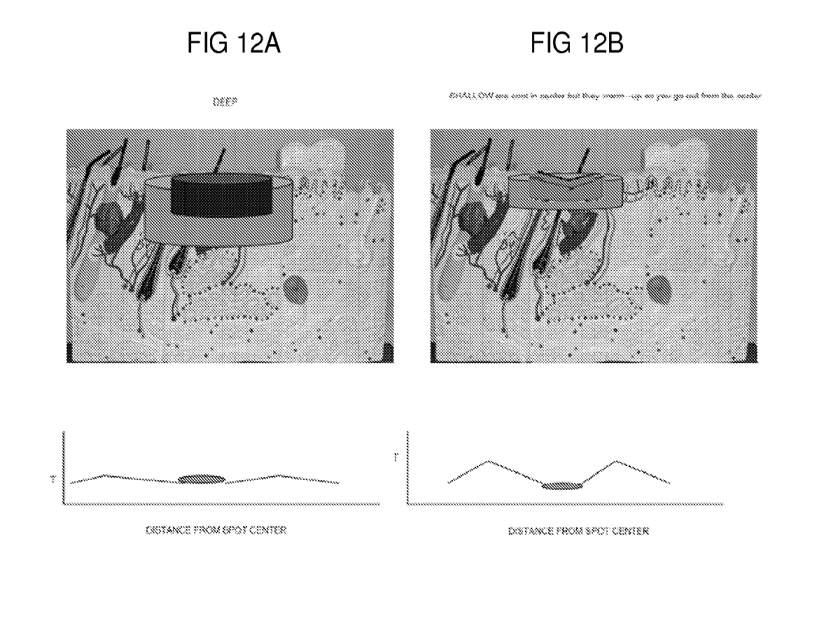

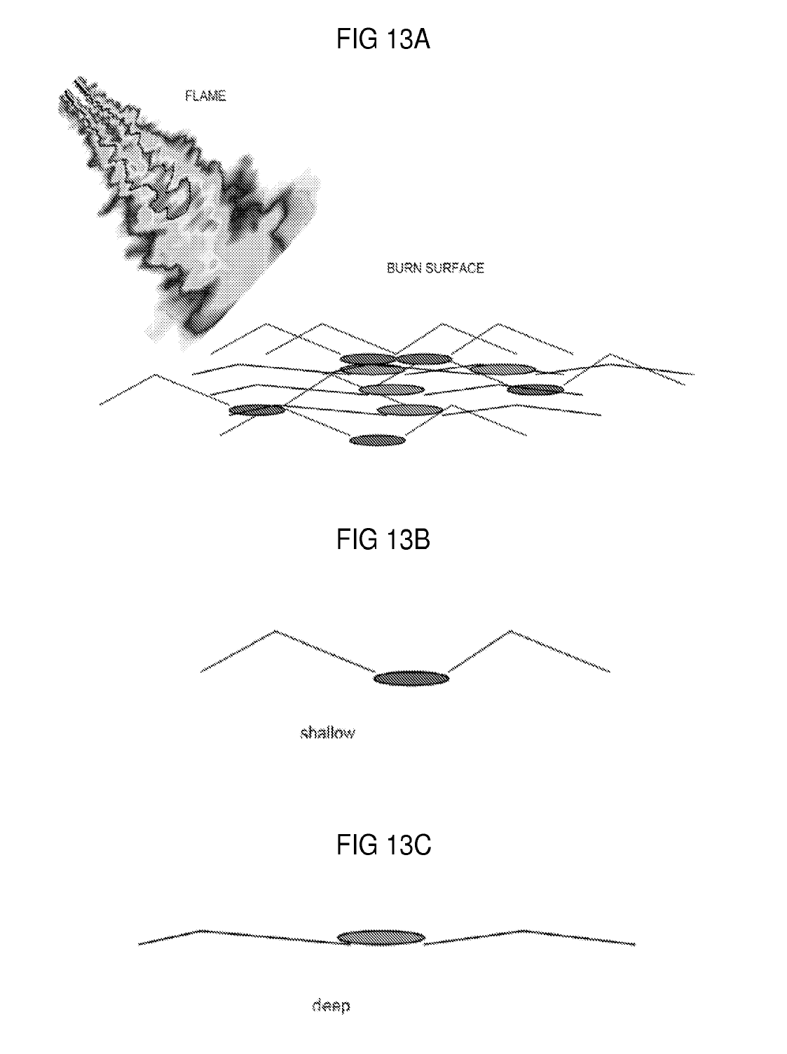

FIG. 12 provides a model for real burns based on infrared contrast imaging (IRCI) data, illustrating a shallower pattern of IR changes radiating from the center of a deep burn than for a shallow burn. FIG. 13 provides an example of an analysis of a burned tissue model and illustrates how shallow and deep burns can be identified based on patterns of IR emission (and the extent of change in IR emission from the center of a burn). In some embodiments, an analysis involves finding low emission (e.g., low temperature) spots, analyzing temperature changes in one or more directions radiating from the low spots (e.g., in 1, 2, 3, 4, or more directions, for example NSEW directions, or along any other relative directions), and determining the rate of change (e.g., the slope of one or more radiating vectors or using any other suitable analysis). In some embodiments, the areas with the flattest slopes from the coolest areas will identify regions of deep burns, whereas areas with the greatest slope changes will represent shallow burns. It should be appreciated that areas that overlap can be determined as to degree of scarring with a suitable calibration curve in some embodiments.

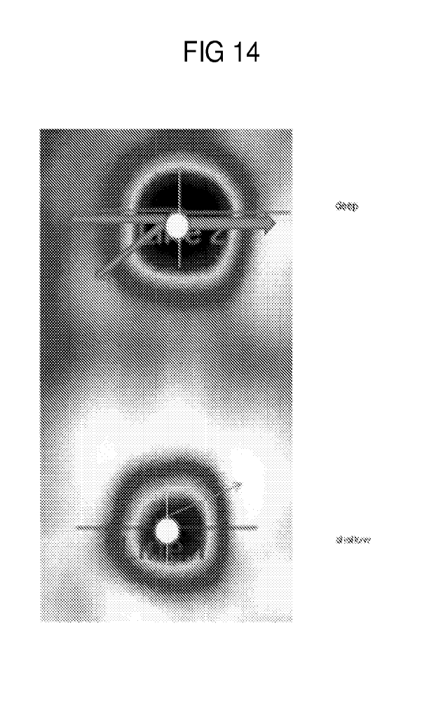

FIG. 14 illustrates vectors representing patterns of IR emission changes from the centers of burned regions that can be used to evaluate burn depth (and, for example, to distinguish shallow burns from deep burns). In some embodiments, steps for evaluating tissue damage, for example burn severity, include one or more of a) finding a center of lowest emission (e.g., lowest temperature), b) measuring a diameter through the coldest area (shallow burns are hotter, and deeper burns are colder); and c) measuring ring areas of IR emissions (e.g., temperatures—shallow burns have higher temperatures and more rings at higher temperatures, whereas deeper burns are colder). Accordingly, in some embodiments, a burn can be evaluated by determining the diameter, surface area, or other measure for one or more (e.g., two or more) different levels of IR emission for different regions of a burn and comparing these measurements to determine the relative burn severity of the different regions. In some embodiments, if skin temperature is measured at around 32° C., then scarring does not occur.

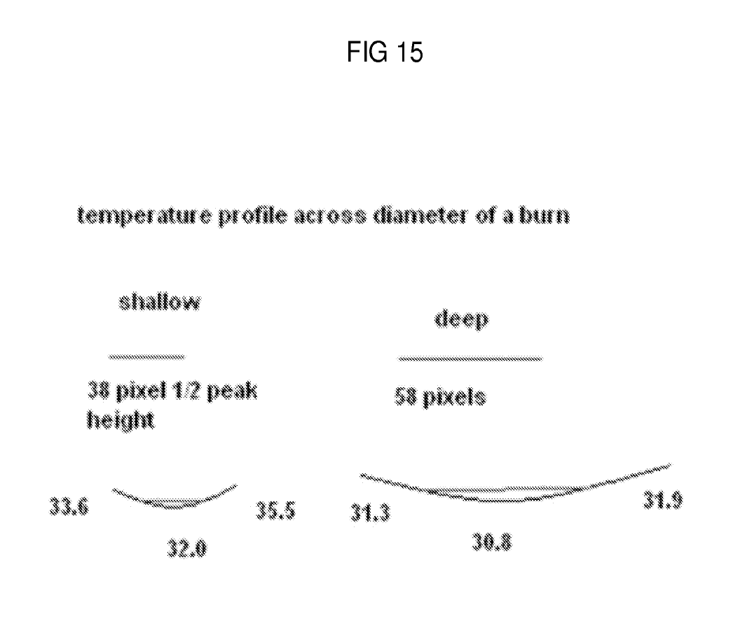

FIG. 15 illustrates non-limiting examples of IR emission profiles (shown as temperature profiles) across the diameter of a burn. This illustrates how IR emission profiles can be used to help evaluate burns early. This is a significant problem, because when a person is burnt, the burn visually looks similar for about two weeks but at the end of that time there are deep burns that scar and shallow burns that heal with minimal to no scarring. Since burns cannot be visually evaluated early on to distinguish regions of deep burn from regions of shallow burn, specific or targeted treatments cannot be applied before the end of two weeks. By that time, a deep burn can be cleaned, but the scarring will be bad. In contrast, an IRCI image of a burn can be used to rapidly (and soon after injury) between deep (on top of figure) and shallow (on bottom of figure) burns. For example, there are distinct differences that are apparent from about 3 hours after the burn to about 48 hours after the burn. After that there are still differences that can be identified, but they are not as clear. In some embodiments, both shallow and deep burns have lowest levels of IR emission (lowest temperatures) at their centers (or in the region of highest tissue damage). However, badly burnt tissue will have lower levels of emission (e.g., a colder middle) and a lower rate of IR emission change (e.g., temperature change) as a function of distance radiating out from the center. In contrast, shallow burns have a higher middle temperature than deep burns (but still lower than normal skin temperature) and the slope of the increase from the center is much greater than for deep burns.

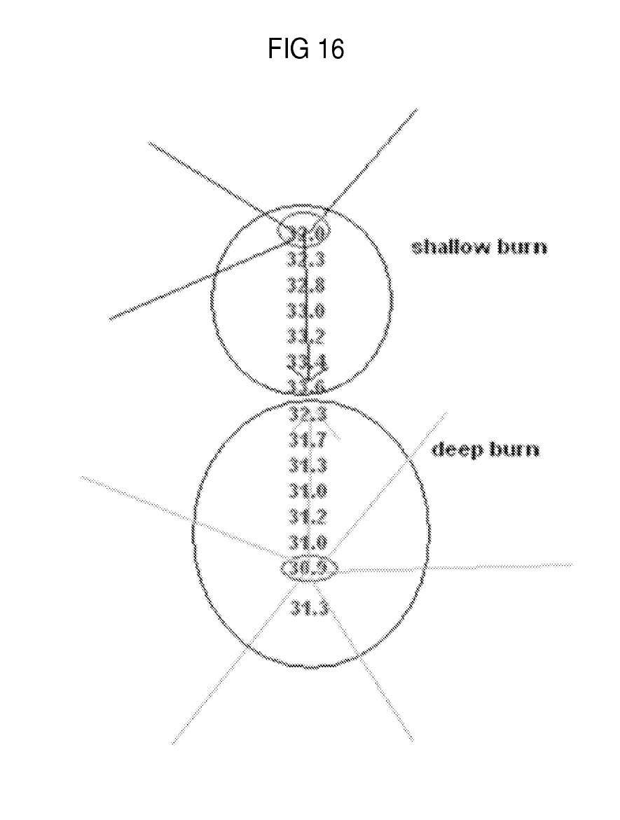

FIG. 16 illustrates non-limiting examples of vectors radiating from the points of lowest emission for shallow and deep burns. In some embodiments, intersections of shallow and deep burns can be determined (e.g., by calculation or image analysis). In some embodiments, this allows for an overlay (e.g., a transparent and/or color overlay) to be prepared (e.g., printed). For example, with one or more physical reference points that can be used to match the images up with the burnt tissue of a subject so that a surgeon or medical practitioner could pick away severely burnt tissue, or otherwise selectively treat tissue as a function of burn severity, under the overlay. In some embodiments, an overlay can be projected or otherwise displayed on the surface of a patient's skin to indicate the relative and/or specific levels of burn for different regions, thereby allowing a surgeon or other medical practitioner to apply different appropriate treatments to different regions based on their burn severity.

This technique of using a printed or projected patterns to assist a physician or medical practitioner can be used for other injuries or conditions (e.g., skin injuries or conditions). In some embodiments, one or more reference points are provided along with the IR pattern to allow the printed or displayed pattern to be aligned with a subject. Different techniques can be used to generate one or more reference points. In some embodiments, a reference point can be a body feature (e.g., a joint or other identifiable body feature). In some embodiments, one or more reference points can be added to the tissue prior to obtaining the IR data. For example, one or more spots of a dye or other material that either increases or reduces an IR signal can be used. In some embodiments, a drop of water can be used to provide a mark due to decrease IR signal. In some embodiments, the drop of water contains a dye or other marker that is left on the skin at the site of the drop. This can be used later to align the printed or displayed pattern by aligning the IR reference spot corresponding to the drop of water with the dye spot on the skin. It should be appreciated that other techniques for producing and using one or more reference points can be used to spatially align or register patterns for treatment as aspects of the invention are not limited in this respect.

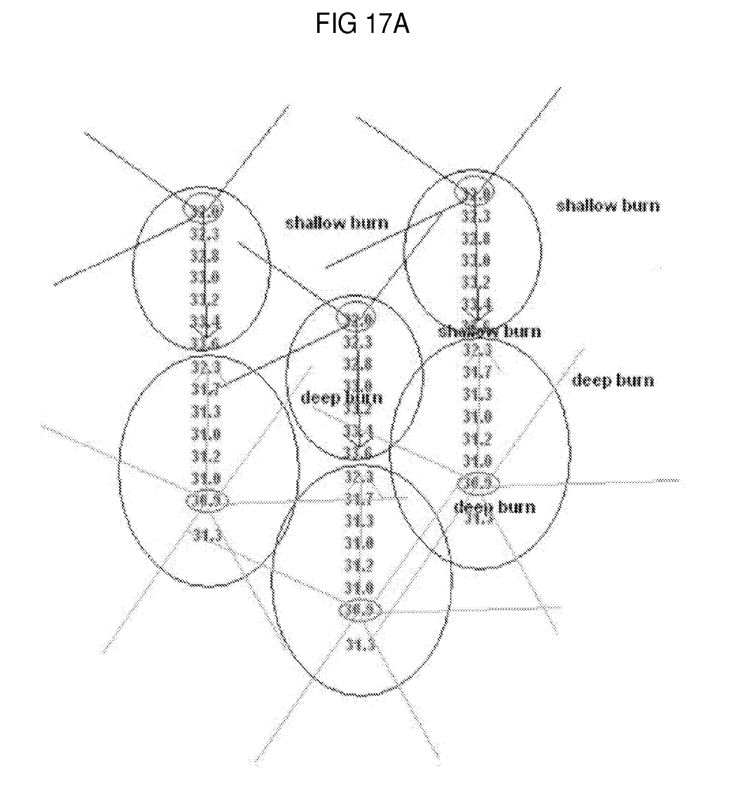

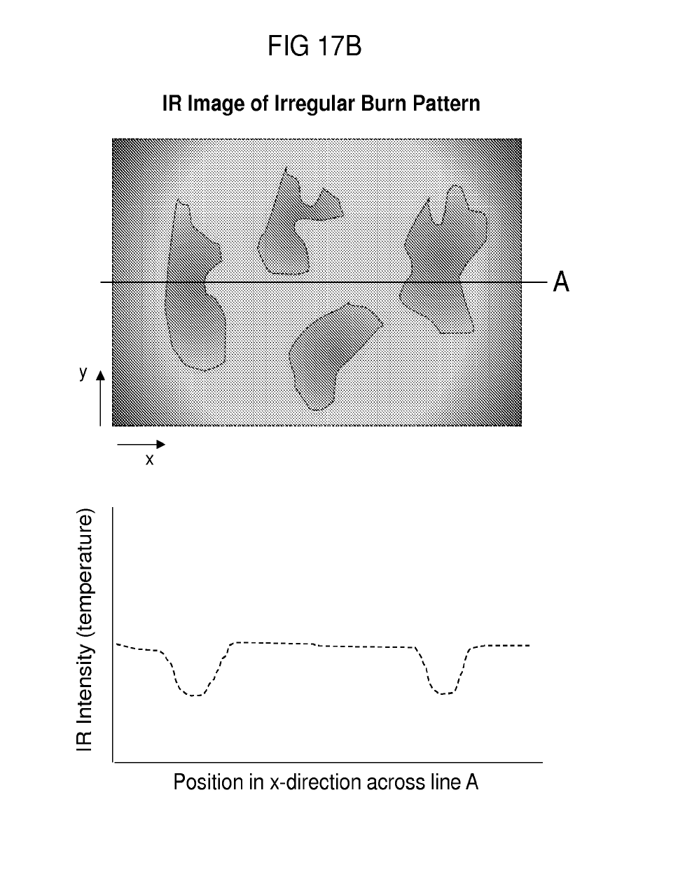

FIG. 17A illustrates further non-limiting examples of vectors radiating from the points of lowest emission for several shallow and deep burns. Accordingly, calculations or other analyses can be performed to create a map of different levels (e.g., different relative levels) of burn. Depending on the extent of a burn and/or the location of a burn, different levels of spatial resolution can be used. For example, FIG. 17B illustrates a non-limiting example of a profile obtained from an IR image containing an irregular burn pattern.

Accordingly, methods described herein can be used to evaluate burned tissue early, for example before visual differences can be identified between shallow and deep burns. This allows for earlier and more effective therapeutic intervention. Similar techniques can be used to evaluate other tissue injuries and/or conditions as described herein.

It should be appreciated that aspects of the invention are based on the natural emissivity of tissue and differences in emissivity caused by injury or disorders. Accordingly, no markers, dyes, or other detectable agents are required in some embodiments. Similarly, Doppler or other acoustic techniques also are not required in some embodiments. However, it also should be appreciated that aspects of the invention can be combined with other techniques that involve Doppler and/or other acoustic techniques and/or detectable markers.

In some embodiments, the levels of tissue emissivity, and, in particular, differences in the levels of emissivity that are associated with different types or degrees of injury can be detected using an infrared detection device that is calibrated to maximize the contrast between different tissue appearances rather than being calibrated to detect the temperature of a tissue. Accordingly, IR contrast imaging can be performed by calibrating an IR detector to be most sensitive to contrasts between different IR levels that are associated with an injury or condition. In some embodiments, a high resolution palette can be used to display different levels of IR emission that may not be distinguishable using typical display palettes that do not provide sufficient contrast (e.g., between IR levels corresponding to physiological injuries or conditions, for example, corresponding to 0-50° C., around 25-50° C.). However, aspects of the invention are not limited in this respect, and information that is obtained using a device (e.g., camera) that is calibrated to detect temperature levels also can be analyzed to evaluate the extent of tissue injury.

In some embodiments, a tissue can be the tissue of a human subject. However, a tissue of an animal (e.g., a vertebrate, invertebrate, mammal, or other subject) can be evaluated using techniques described herein. In some embodiments, the subject has been injured and techniques described herein are used to evaluate the extent of the injury. Accordingly, methods described herein can be used to determine whether and/or how to treat a subject based on the presence or extent of injuries detected. In some embodiments, the tissue is skin. However, in some embodiments, the tissue is mucosal tissue, eye tissue (e.g., corneal tissue, or other ocular tissues), or an internal tissue such as, fascia or another connective tissue.

In some embodiments, a pattern of differences in infrared emissions can be used to establish an extent and/or pattern of tissue injury. This information can be used to determine a course of therapy for the injured tissue. In some embodiments, different types of therapy may be recommended based on the level or extent of injury.

In some embodiments, a pattern of injury (e.g., based on an infrared pattern) of a tissue that is determined using techniques described herein can be displayed on a subject in order to precisely target a therapy to the appropriate tissue region. In some embodiments, different therapies are applied or targeted to different regions of injured tissue based on the level of injury determined using techniques described herein. By displaying the pattern of injury on the subject (e.g., using a projector, or other technique), levels or types of treatment can be precisely targeted. However, it should be appreciated that instead of directly displaying a pattern on the subject, the pattern can be displayed on a screen or photo or other medium that a physician can use to select and/or target one or more treatments to the appropriate injury regions.

In some embodiments, one or more physical reference points are provided along with the injury pattern. Reference points can be body regions (e.g., joints such as elbows, knees, or hips, extremities such as fingers or toes, the head or features on the head such as the nose or ears, or other body parts). In some embodiments, one or more physical markers can be applied to body regions (e.g., colored markers, water spots, other liquid spots, etc.) near the site of an injury and the position of the marker(s) relative to the injury pattern can be determined and used in therapy. For example, one or more natural or artificial reference points can be used to precisely locate on a subject the different levels of injury that were identified using methods described herein. This can be useful for targeting therapy, for example, in instances where large extents of tissue are injured (e.g., burned) and it can be difficult to determine how to treat different parts of the injured (e.g., burned regions).

In some embodiments, patterns of injury can be determined based on the contrast (e.g., infrared contrast, for example, far infrared contrast) between different parts of an injured tissue. In some embodiments, it is not the absolute level of emission that is used to determine injury patterns, rather the differences in emission between different regions are useful to determine relative levels of injury. However, in some embodiments, reference levels of emission can be established for normal and/or different levels of injury. It should be appreciated that reference levels may be different for different parts of the body and for different individuals (e.g., based on different ages and/or other physical traits). However, as discussed above, the relative emission levels for any individual can be used to determine injury patterns. In some embodiments, a normal level can be established for an individual based on levels of emission detected for undamaged tissue in the individual. Accordingly, an emission pattern can be established for a region expected to be injured and also for surrounding regions. In some embodiments, the pattern is detected for a large region in order to ensure that non-injured tissue is included in the analysis. However, non-injured tissue is not always required. As discussed herein, the contrast between different tissue regions can be used to determine a pattern of injury.

In some embodiments, different emissions (e.g., different infrared emissions) from a tissue region can reflect surface tissue damage. However, it also should be appreciated that tissue damage at different depths from the tissue surface also can change the emission patterns (e.g., infrared emission patterns). Accordingly, patterns of emission changes (e.g., contrasts in emission levels) can be used to evaluate surface and or deeper tissue injuries. In some embodiment, the techniques herein are used to evaluate the extent of an injury to skin or another tissue. For example, in some embodiments, the techniques are used to determine the depth of a skin injury, e.g., whether a skin injury is limited to epidermal tissue or extends into or through dermal tissue or into or through basement membrane.

In some embodiments, aspects of the invention are useful for detecting injuries and/or evaluating the extent and/or severity of an injury before other physical signs (e.g., visual signs such as bruising, blistering, etc., or any combination thereof) are detectable. This allows injuries to be detected and/or evaluated earlier. These injuries can therefore be treated earlier. This can provide significant benefits associated with early treatment and/or triage of patients after an accident or other traumatic event.

In certain embodiments, aspects of the invention allow for more precise treatments to be targeted to different regions of an injury based on different levels of injury detected using infrared emission patterns detected from the tissue.

Accordingly, in some embodiments a method of evaluating, or assisting in the evaluation of an injury (e.g., a burn) in a subject includes detecting infrared emission from a tissue region (e.g., a burn) in a subject, and determining a pattern of infrared emission levels for the tissue region (e.g., burn).

In some embodiments, the infrared emission is detected less than 72 hours (e.g., less than 48 hours, less than 24 hours, less than 12 hours, less than 6 hours, less than 3 hours, less than 1 hour, less than 30 minutes, less than 15 minutes, less than 10 minutes, or less than 5 minutes) after an event that caused the tissue injury (e.g., burn) in the subject. However, it should be appreciated that techniques described herein are not limited to detecting short term tissue damages or changes, but can also be used to detect and evaluate long term damage and/or tissue changes.

In some embodiments, the injury should be cooled prior to obtaining IR data. For example, if the injury is associated with heat (e.g., a burn), the heat from the injury itself (e.g., the heat of the burn itself) can obscure the IR patterns or profiles associated with changes in the emissivity of the tissue caused by the injury. For example, a solution or saline could be applied to the area of suspected of being injured prior to obtaining IR data. The solution could be water or a saline solution or any other solution at any suitable temperature (e.g., cold, cool, room temperature, or body temperature, for example).

In some embodiments, the event that caused the tissue burn was an exposure to heat, an exposure to a chemical element, or an exposure to radiation. In some embodiments, the tissue burn is a skin burn.

In some embodiments, infrared emission is detected from a tissue region that includes injured (e.g., burnt) tissue and tissue surrounding the injured tissue, and wherein the pattern of infrared emission levels includes both the injured tissue and the surrounding tissue. In some embodiments, the surrounding tissue includes a region of tissue that is at the margin of the injured (e.g., burnt) tissue and that is at least 0.5 cm, 1 cm, 5 cm, or more wide.

In some embodiments, the infrared radiation emission that is detected is a far infrared radiation emission. In some embodiments, the far infrared wavelengths detected are 15-1,000 μm.

In some embodiments, the pattern is a two-dimensional pattern of emission levels, e.g., a pattern of emission levels measured over time across an area. In some embodiments, the pattern is a three-dimensional pattern of emission levels, e.g., a pattern of emission levels measured over time from a volume, or a pattern of emission levels within a volume of tissue.

In some embodiments, a method of evaluating a burn further comprises evaluating the emission pattern to identify one or more different levels of burn severity within the tissue burn. In some embodiments, the different levels of burn severity correspond to different burn degrees. In some embodiments, the different levels of burn severity correspond to different burn depths. In some embodiments, the different levels of burn severity are indicative of different degrees of tissue injury associated with different therapeutic treatments. In some embodiments, the different levels of burn severity are discrete levels. In some embodiments, the different levels of burn severity represent a continuous gradation of burn severity.

In some embodiments, an infrared emission pattern is indicative of, a) a heat pattern within the injured (e.g., burnt) tissue; b) a pattern of density variation within the tissue (e.g., vascular density); c) a hydration pattern within the injured (e.g., burnt) tissue, and/or; d) a pattern of tissue surface variation over the injured (e.g., burnt) tissue.

In some embodiments, the pattern of density variation represents density changes associated with the burnt tissue. In some embodiments, the pattern of tissue surface variation represents tissue surface changes associated with the burnt tissue.

In some embodiments, the slope of infrared emission change between a first region in the injured (e.g., burnt) tissue and a second region in the tissue (e.g., injured tissue) is determined. In some embodiments, the first region is a region of low emission. In some embodiments, a profile of relative infrared emission levels is determined across a line segment (e.g., a stretch of connected pixels) spanning a portion of 2D or 3D image of a tissue or surface, e.g., as depicted in FIG. 3. In some embodiments, the line segment spans multiple regions of the tissue or surface having different infrared emission characteristics and the different characteristics are analyzed to identify areas of damaged tissue or surface aberrations of an object.

In some embodiments, an appropriate therapeutic treatment is recommended/selected based on the infrared emission pattern of the injured (e.g., burnt) tissue. In some embodiments, an appropriate therapeutic treatment is administered based on the infrared emission pattern of the injured (e.g., burnt) tissue. In some embodiments, a first therapeutic treatment is recommended/selected for a first region of the injured (e.g., burnt) tissue and a second therapeutic treatment is recommended/selected for a second region of the injured (e.g., burnt) tissue, based on different levels of infrared emission from the first and second regions of the injured tissue.

In some embodiments, a first therapeutic treatment is selected for a first region of the burnt tissue and a second therapeutic treatment for a second region of the burnt tissue, based on different levels of infrared emission from the first and second regions of the burnt tissue.

In some embodiments, a therapeutic treatment comprises removing burnt tissue. In some embodiments, the first therapeutic treatment comprises removing tissue from the first region of burnt tissue. In some embodiments, the depth of burnt tissue that is removed is based on the level of infrared emission. In some embodiments, the depth of tissue that is removed from the first region is based on the level of infrared emission from the first region.

In some embodiments, the first therapeutic treatment comprises administering a first compound to the first injured (e.g., burnt) region. In some embodiments, the second therapeutic treatment comprises administering a second compound to the second injured (e.g., burnt) region.

In some embodiments, first and second compounds (and/or first and second levels of a similar or identical compound) are provided on a solid support (e.g., a bandage) and the pattern of the first and second compounds on the bandage corresponds to the pattern of the first and second injured (e.g., burnt) regions on the subject. It should be appreciated that more than two levels or types of therapeutic compounds can be provided on a solid support in a pattern that corresponds to a pattern of more than two levels of tissue injury detected for a subject.

In some embodiments, aspects of the invention relate to a bandage comprising a first region having a first therapeutic compound for treating an injury (e.g., a burn), and a second region having a second therapeutic compound for treating an injury (e.g., a burn), wherein the first and second regions are configured in a pattern on the bandage that corresponds to a pattern of different levels of injured tissue associated with a subject. In some embodiments, the bandage comprises a pattern of three or more different regions each having a different therapeutic compound, wherein the pattern of the different therapeutic compounds on the bandage corresponds to a pattern of different levels of injured tissue associated with an injury on a subject.

In some embodiments, aspects of the invention relate to a bandage (or other medical application), that can have a pattern of burn/wound severity printed out on at least one surface corresponding to a particular burn or wound so that it can be placed on the burn or wound. Reference points can be marked on a patient and used to align the printed bandage. In some embodiments, the bandage can have a decellularizing ink (e.g., SDS, or other decellularizing compound or mixture) or other debriding composition that can be printed in a concentration matching the severity of the burn or wound. Accordingly, this can dissolve tissue in a specific pattern that corresponds to the burn or wound severity. In some embodiments, a bandage will trap softened tissue in its structure and, when removed, take tissue off in proportion to the severity of the burn or wound and its spatial locations. In some embodiments, another bandage can be printed with a treatment and/or cells to be delivered to specific portions of a burn. In some embodiments, a separate device can be used to spray or otherwise deliver treatments and/or graft cells onto exposed areas. In some embodiments, physical reference points can be used to align bandages (or target other treatments) as a function of levels of tissue damage as determined by IR analysis.

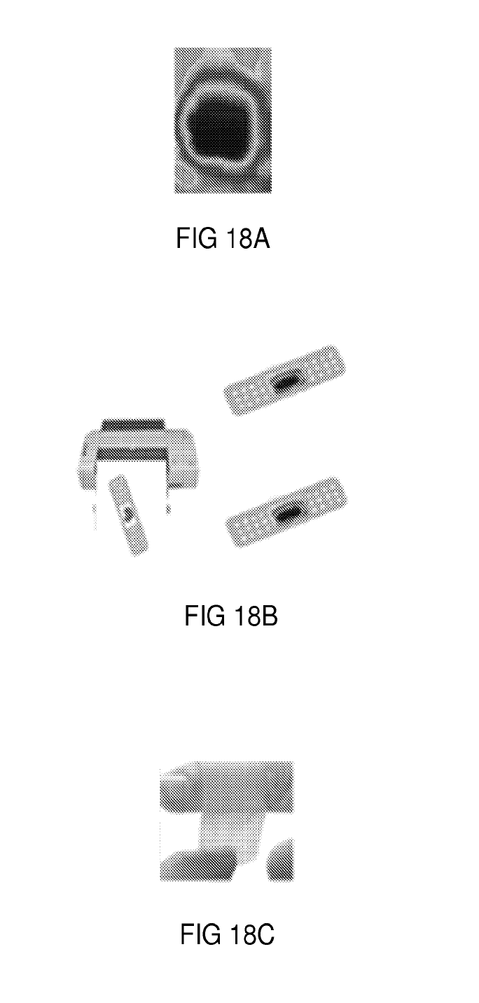

Similarly, other treatments (e.g., one or more antibiotics, antimicrobials, anti-inflammatories, cytotoxic, anti-cancer, anti-oncogenic, palliative, oxidizing agents, peroxide, hydrogen peroxide, therapeutic drugs, neomycin, or other compounds, or combinations thereof) can be printed on bandages in one or more spatial patterns that correspond to IR patterns (e.g., as illustrated in FIG. 18) in order to treat wounds, burns, infections, frostbite, gangrene, skin cancer, skin disorders (e.g., a callus or a corn) or other conditions. In some embodiments, different compositions are provided on a single bandage (or other support material). In some embodiments, different levels (e.g., amounts or concentrations) of one or more compositions are provided on a single bandage (or other support material) in a pattern that corresponds to an injury, disease, or disorder pattern (e.g., corresponding to the extent and/or severity). In some embodiments, a printer can be used to print multiple or single preparations in a spatial relationship that corresponds to an IR pattern or other imaging techniques. These can be used to provide spatially accurate images of damage and/or provide treatments in spatially accurate position to prepare, treat, and/or heal different levels of injury simultaneously. Accordingly, in some embodiments, a method of producing a bandage comprising a spatial configuration of one or more compositions comprises obtaining IR data for a target tissue (e.g., injured or diseased), processing the data to determine a pattern of compositions or levels of composition (e.g., treatment) that are adapted to the pattern of the tissue (e.g., skin) injury or disorder determined from the IR data, and preparing a bandage or other support with the pattern of compositions or levels of composition (e.g., using a printer).

In some embodiments, water, biological solutions (e.g., blood, saliva, etc., or any combination thereof), buffers, etc., or any combination thereof can be used as external coverings or coatings for a wound or burn, for example, to enhance the detection or different levels of IR emission (e.g., from hot or cold areas) or differences within an area.

In some embodiments, the pattern of infrared emission levels will be used as a virtual image to monitor the depth and extent of burn wound debridement.

In some embodiments, the pattern of infrared emission levels will be used as a virtual image to monitor the depth and extent of burn injury in a clinical trial.

In some embodiments, project analysis information on wound can be aligned with reference points marked on patient. Treatment can be dictated by the spatially accurate and analyzed data.

Aspects of methods for evaluating IR image data disclosed herein may be implemented in any of numerous ways. For example, the various methods or processes outlined herein may be coded as software that is executable on one or more processors that employ any one of a variety of operating systems or platforms. Such software may be written using any of a number of suitable programming languages and/or programming or scripting tools, and also may be compiled as executable machine language code or intermediate code that is executed on a framework or virtual machine. The MATLAB image processing toolbox (The MathWorks, Inc., Natick, Mass.) is an exemplary, but non-limiting, system that may be used for implementing certain aspects of the methods disclosed herein.

In this respect, aspects of the invention may be embodied as a computer readable medium (or multiple computer readable media) (e.g., a computer memory, one or more floppy discs, compact discs, optical discs, magnetic tapes, flash memories, circuit configurations in Field Programmable Gate Arrays or other semiconductor devices, or other tangible computer storage medium) encoded with one or more programs that, when executed on one or more computers or other processors, perform methods that implement the various embodiments of the invention discussed herein. The computer readable medium or media can be transportable, such that the program or programs stored thereon can be loaded onto one or more different computers or other processors to implement various aspects of the present invention as discussed above.

The terms “program” or “software” are used herein in a generic sense to refer to any type of computer code or set of computer-executable instructions that can be employed to program a computer or other processor to implement various aspects of the present invention as discussed above. Additionally, it should be appreciated that according to one aspect of this embodiment, one or more computer programs, which when executed perform certain methods disclosed herein, need not reside on a single computer or processor, but may be distributed in a modular fashion among or between a number of different computers or processors to implement various aspects of the present invention.

Computer-executable instructions may be in many forms, such as program modules, executed by one or more computers or other devices. Generally, program modules include routines, programs, objects, components, data structures, etc. that perform particular tasks or implement particular abstract data types. Typically the functionality of the program modules may be combined or distributed as desired in various embodiments.

1. A method of evaluating a burn, a wound, or a natural biological state of one or more cells, tissues, organs, organ systems, or objects, the method comprising:
detecting infrared emission from a tissue burn/wound, cell, tissue, organ, system, or object; and,
determining a pattern of infrared emission levels.

2-40. (canceled)
